SEQUENCE LISTING (17 sequences)

SEQ ID NO: 1 - DNA, 2364 bp, Stevia rebaudiana
atgaagaccggcttcatctctcccgccaccgtcttccaccaccgtatttctccggcaaccaccttccgccaccacctttctccggcgaccaccaactccactggaattgtagctcttagagacatcaacttccggtgtaaagcggtatccaaagagtactctgatttactacaaaaagatgaggcttcatttaccaagtgggacgatgacaaagtgaaggaccatttggacacaaataagaatttgtatccaaacgatgagatcaaggagtttgttgagagcgtgaaagcaatgtttggttctatgaatgacggagaaataaatgtgtcagcgtatgatacggcttgggttgcactcgtgcaagatgttgatggaagtggttcccctcaatttccatcaagtttggagtggatcgcgaacaatcaactctcagatgggtcttggggcgatcatttgttattttcggctcatgataggatcattaacacgttggcatgtgttatagcgcttacttcttggaacgtccatccaagtaaatgtgaaaaaggactgaattttcttagagaaaacatatgtaaactcgaagacgagaacgcggaacatatgccaattggttttgaagtcacgttcccgtcgctaatagatatcgcaaagaagctaaatattgaagttcctgaggatactcctgccttaaaagaaatttatgcaagaagagacataaaactcacaaagataccaatggaagtattgcacaaagtgcccacaactttacttcatagtttggaaggaatgccagatttggaatgggaaaaacttctgaaattgcaatgcaaagatggatcatttctgttttctccatcatctactgcttttgcactcatgcaaacaaaagatgaaaagtgtcttcagtatttgacaaatattgttaccaaattcaatggtggagttccgaatgtgtacccggtggatctattcgaacatatttgggtagttgatcgacttcaacgacttgggattgctcgttatttcaaatcagagatcaaagattgcgttgaatatattaacaagtattggacaaagaatgggatttgttgggcaagaaacacgcacgtacaagatattgatgataccgcaatgggatttagggttttaagagcacatggttatgatgttactccagatgtatttcgacaatttgagaaggatggtaaattcgtatgtttcgctggacagtcaacacaagccgtcaccggaatgttcaatgtgtatagagcgtcacaaatgctctttcccggagaaagaattcttgaagatgcaaagaaattttcatataattatttgaaagaaaaacaatcgacaaatgagcttcttgataaatggatcatcgccaaagacttacctggagaggttggatatgcgctagacataccatggtatgcaagcttaccgcgactcgagacaagatattacttagagcaatacgggggcgaggatgatgtttggattggaaaaactctatacaggatgggatatgtgagcaataatacgtaccttgaaatggccaaattggactacaataactatgtggccgtgcttcaactcgaatggtacactatccagcaatggtatgttgatatcggtatcgaaaagtttgaaagtgacaatatcaaaagcgtattagtgtcgtattacttggctgcagccagcatattcgagccggaaaggtccaaggaacgaatcgcgtgggctaaaaccaccatattagttgacaagatcacctcaatttttgattcatcacaatcctcaaaagaggacataacagcctttatagacaaatttaggaacaaatcgtcttctaagaagcattcaataaatggagaaccatggcacgaggtgatggttgcactgaaaaagaccctacacggcttcgctttggatgcactcatgactcatagtcaagacatccacccgcaactccatcaagcttgggagatgtggttgacgaaattgcaagatggagtagatgtgacagcggaattaatggtacaaatgataaatatgacagctggtcgttgggtatccaaagaacttttaactcatcctcaataccaacgcctctcaaccgtcacaaatagtgtgtgtcacgatataactaagctccataacttcaaggagaattccacgacggtagactcgaaagttcaagaactagtgcaacttgtgtttagcgacacgcccgatgatcttgatcaggatatgaaacagacgtttctaaccgtcatgaaaaccttctactacaaggcgtggtgtgatccgaacacgataaatgaccatatctccaaggtgttcgagattgtaatatga

SEQ ID NO: 2 - protein, 787 aa, Stevia rebaudiana
MKTGFISPATVFHHRISPATTFRHHLSPATTNSTGIVALRDINFRCKAVSKEYSDLLQKDEASFTKWDDDKVKDHLDTNKNLYPNDEIKEFVESVKAMFGSMNDGEINVSAYDTAWVALVQDVDGSGSPQFPSSLEWIANNQLSDGSWGDHLLFSAHDRIINTLACVIALTSWNVHPSKCEKGLNFLRENICKLEDENAEHMPIGFEVTFPSLIDIAKKLNIEVPEDTPALKEIYARRDIKLTKIPMEVLHKVPTTLLHSLEGMPDLEWEKLLKLQCKDGSFLFSPSSTAFALMQTKDEKCLQYLTNIVTKFNGGVPNVYPVDLFEHIWVVDRLQRLGIARYFKSEIKDCVEYINKYWTKNGICWARNTHVQDIDDTAMGFRVLRAHGYDVTPDVFRQFEKDGKFVCFAGQSTQAVTGMFNVYRASQMLFPGERILEDAKKFSYNYLKEKQSTNELLDKWIIAKDLPGEVGYALDIPWYASLPRLETRYYLEQYGGEDDVWIGKTLYRMGYVSNNTYLEMAKLDYNNYVAVLQLEWYTIQQWYVDIGIEKFESDNIKSVLVSYYLAAASIFEPERSKERIAWAKTTILVDKITSIFDSSQSSKEDITAFIDKFRNKSSSKKHSINGEPWHEVMVALKKTLHGFALDALMTHSQDIHPQLHQAWEMWLTKLQDGVDVTAELMVQMINMTAGRWVSKELLTHPQYQRLSTVTNSVCHDITKLHNFKENSTTVDSKVQELVQLVFSDTPDDLDQDMKQTFLTVMKTFYYKAWCDPNTINDHISKVFEIVI

SEQ ID NO: 3 - DNA, 841 bp, Artificial Sequence
  Description of Artificial Sequence Synthetic polynucleotide
agattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggac

SEQ ID NO: 4 - DNA, 356 bp, Artificial Sequence
  Description of Artificial Sequence Synthetic polynucleotide
ttttaatgtttagcaaatgtcctatcagttttctctttttgtcgaacggtaatttagagttttttttgctatatggattttcgtttttgatgtatgtgacaaccctcgggattgttgatttatttcaaaactaagagtttttgcttattgttctcgtctattttggatatcaatcttagttttatatcttttctagttctctacgtgttaaatgttcaacacactagcaatttggctgcagcgtatggattatggaactatcaagtctgtgggatcgataaatatgcttctcaggaatttgagattttacagtctttatgctcattgggttgagtataatatagtaaaaaaatagg

SEQ ID NO: 5 - DNA, 1422 bp, Stevia rebaudiana
atgtctttgagctatctatctccaacacaaaccaatctaatcactttctccgacacctgcaaatcccaaacccaccttctcaagctccaaggtgggttttgcttcaagagaaaagatgttaagctcgcaggaaaagggattcgatgttcggcgcagcctccgccgccgccggcgtggccgggaacggcgctggttgaccccgggacgaagaattgggacggccctaaacctatttcaatagtgggatctactggttcaattgggactcagacacttgatattgttgctgaaaaccctgataagtttcgagttgtagcacttgctgctggatcaaacgtgactcttcttgctgaacagataaaggcattcaaaccacaattagtttcaatccagaacgaatctttagttggcgaacttaaagaagcattagctgatgctgattacatgcctgaaattattcccggagatcaaggcatcattgaggtcgctcgccatcccgattgtgtcactgttgtcacaggcatagttggttgtgctggtttgaagcctacagttgctgccattgaagcagggaaaaacatagcattagctaataaagaaaccctaattgccggtggtccgttcgttcttcctcttgcacgtaaacataatgttaaaattcttcctgctgattcagaacattctgctatattccagtgtattcaaggctttcctgaaggtgctttgaggcgtataatcttaaccgcatctggtggtgcttttagagatttaccagttgaaaaactaaaagatgttaaagtagccgatgcattaaaacatccaaactggagtatgggtaaaaaaatcacggttgattcagcgacacttttcaacaagggtcttgaagttatcgaagctcattatctttacgggtcagattatgataatattgaaattgttattcatcctcaatctatcatacactccatggttgagacacaggactcttcggttctagcccaattaggttggcccgatatgcgtttgccaattctttacacgttatcttggcccgatagaatatcatgttctgaaattacttggcctcgcctcgatctttgcaagttgggatcattaacatttaaagctcccgataatgtgaaatacccgtcgatggatttggcttatgccgctggacgagctggcggcacgatgaccggagttcttagtgccgccaatgagaaagcggttgagatgttcattgatgaaaagattcaatatttggacatatttaaagttgttgagctaacatgtgcgaaacatcaatccgaactcgtaactgcaccgtcacttgaagaaatcgtgcattatgacttgtgggctcgtgattatgcggctagtttgaagtcatcacccggtttgaccgcggtagctcttgtatga

SEQ ID NO: 6 - protein, 473 aa, Stevia rebaudiana
MSLSYLSPTQTNLITFSDTCKSQTHLLKLQGGFCFKRKDVKLAGKGIRCSAQPPPPPAWPGTALVDPGTKNWDGPKPISIVGSTGSIGTQTLDIVAENPDKFRVVALAAGSNVTLLAEQIKAFKPQLVSIQNESLVGELKEALADADYMPEIIPGDQGIIEVARHPDCVTVVTGIVGCAGLKPTVAAIEAGKNIALANKETLIAGGPFVLPLARKHNVKILPADSEHSAIFQCIQGFPEGALRRIILTASGGAFRDLPVEKLKDVKVADALKHPNWSMGKKITVDSATLFNKGLEVIEAHYLYGSDYDNIEIVIHPQSIIHSMVETQDSSVLAQLGWPDMRLPILYTLSWPDRISCSEITWPRLDLCKLGSLTFKAPDNVKYPSMDLAYAAGRAGGTMTGVLSAANEKAVEMFIDEKIQYLDIFKVVELTCAKHQSELVTAPSLEEIVHYDLWARDYAASLKSSPGLTAVALV

SEQ ID NO: 7 - DNA, 2148 bp, Stevia rebaudiana
atggcggtggcaggatcgaccatgaacctgcatctcacttcatctccatacaagacagttccatcactctgtaaattcaccagaaaacagttccgattaaaggcctctgcaacgaatccagacgctgaagatgggaagatgatgtttaaaaacgataaacccaatttgaaggtcgaattcactggggagaaaccggtgacaccattactggataccattaattaccctgtgcacatgaaaaacctcaccactcaggatcttgagcaattagcagcagaacttagacaagatattgtatattcagtagcgaatacaggtggtcatttgagttcaagtttaggtgttgttgaattgtctgttgctttacaccatgttttcaacaccccagatgacaagatcatttgggacgttggtcaccaggcatacccacataagattttgaccggaagaaggtcaaagatgcacaccataagaaaaacttctggtttagctggttttcctaaacgagatgaaagtgctcatgatgcttttggtgctggacatagttctacaagcatctctgctggcctaggtatggctgtcggtagagatttattagggaaaaccaacaacgtgatatcggtgatcggagatggcgccatgacggccggacaagcatatgaggcgatgaataatgcaggatttcttgattcaaatctaatcgtcgttttaaacgacaacaagcaagtttcattaccgactgccacgttggacggacctgcaactcccgtcggggctctcagcggcgctttatccaaattgcaagccagtaccaagttccggaagcttcgtgaagccgccaagagcattactaaacaaattggacctcaagcacatgaagtggcggcgaaagtcgacgaatacgcaagaggtatgattagtgctagcgggtcgactttattcgaggagctcggattatactacatcggtcccgtcgatggtcacaatgttgaagatttagtcaacatttttgaaaaagtcaagtcaatgcccgcacccggaccggttctaatccacatcgtgaccgaaaaaggcaaaggttaccctcctgctgaagccgctgctgaccgcatgcacggagttgtgaagtttgatgttccaactggaaaacaattcaagacaaaatcaccgacactttcgtatactcagtattttgctgaatcacttataaaagaagctgaagctgataacaagattgtcgcgatacacgccgccatgggaggcggtaccggactcaattacttccagaagaagtttccggaacgttgttttgacgtcggtatcgcggaacaacacgcagttactttcgccgcgggtttagccaccgaaggtcttaaaccattttgcgcgatctattcgtcgtttttgcaacgaggatacgatcaagtggtgcatgatgttgatctacaaaagttaccggttcggtttgcgatggaccgagctggtttagtcggggctgatggaccgacacattgtggtgcgtttgacataacctacatggcgtgtctaccaaacatggtggtgatggctccagccgatgaagccgaattgatgcacatggttgcaacggctgcagccattgacgacagaccgagttgctttcggttcccaagaggcaatggcattggtgcaccacttcctcctaataacaaagggattcccatagaggttggtaaaggaagaatattacttgaaggaactcgagttgcgatattgggatacggttcgatagttcaagaatgtctaggtgcggctagcttgcttcaagcccataacgtgtctgcaaccgtagccgatgcgcggttctgcaaaccgttagacaccggactgattagacgattagccaacgagcatgaagtcttacttaccgtagaggaaggctcgattggtggatttggatcacacgttgctcactttctaagcttaaatggtctcttagatggaaaacttaagcttagagcaatgactcttcctgataaatacattgatcatggtgcaccacaagatcagcttgaagaagccggtctttcttcaaaacatatttgttcatctcttttatcacttttgggaaaacctaaagaagcacttcaatacaaatcaataatgtaa

SEQ ID NO: 8 - protein, 715 aa, Stevia rebaudiana
MAVAGSTMNLHLTSSPYKTVPSLCKFTRKQFRLKASATNPDAEDGKMMFKNDKPNLKVEFTGEKPVTPLLDTINYPVHMKNLTTQDLEQLAAELRQDIVYSVANTGGHLSSSLGVVELSVALHHVFNTPDDKIIWDVGHQAYPHKILTGRRSKMHTIRKTSGLAGFPKRDESAHDAFGAGHSSTSISAGLGMAVGRDLLGKTNNVISVIGDGAMTAGQAYEAMNNAGFLDSNLIVVLNDNKQVSLPTATLDGPATPVGALSGALSKLQASTKFRKLREAAKSITKQIGPQAHEVAAKVDEYARGMISASGSTLFEELGLYYIGPVDGHNVEDLVNIFEKVKSMPAPGPVLIHIVTEKGKGYPPAEAAADRMHGVVKFDVPTGKQFKTKSPTLSYTQYFAESLIKEAEADNKIVAIHAAMGGGTGLNYFQKKFPERCFDVGIAEQHAVTFAAGLATEGLKPFCAIYSSFLQRGYDQVVHDVDLQKLPVRFAMDRAGLVGADGPTHCGAFDITYMACLPNMVVMAPADEAELMHMVATAAAIDDRPSCFRFPRGNGIGAPLPPNNKGIPIEVGKGRILLEGTRVAILGYGSIVQECLGAASLLQAHNVSATVADARFCKPLDTGLIRRLANEHEVLLTVEEGSIGGFGSHVAHFLSLNGLLDGKLKLRAMTLPDKYIDHGAPQDQLEEAGLSSKHICSSLLSLLGKPKEALQYKSIM

SEQ ID NO: 9 - DNA, 1431 bp, Stevia rebaudiana
atgattcaagttctaacaccgatcctcctcttcctcattttcttcgttttctggaaggtttacaagcaccagaaaaccaaaatcaatcttccaccgggaagcttcggatggccatttctgggcgaaactctggcacttctacgtgcaggttgggattcagagccggagagatttgttcgtgaacggatcaagaaacacggaagtcctctagtgtttaagacgtcgttgtttggcgaccattttgcggtgttgtgtggacctgccggaaacaagttcctgttctgcaacgagaacaagctggtggcgtcgtggtggccggttccggtgaggaagcttttcggcaagtctctgctcacgattcgtggtgatgaagctaagtggatgaggaagatgttgttatcgtatcttggtcctgatgctttcgcaactcattatgccgtcacaatggatgtcgtcacccgtcggcatatcgacgttcattggcgagggaaagaagaggtgaacgtattccaaaccgttaagttatatgcctttgagcttgcatgtcgtttattcatgaacctagacgacccaaaccacattgcaaaactcggttccttgttcaacatttttttgaaaggcatcattgagcttccaatcgacgtcccagggacacgattttatagctccaaaaaagcaggagcagctatcaggattgaactaaaaaaattgattaaagcaagaaaactggaactgaaagaagggaaggcatcatcttcacaagacctcttatcacatttgcttacatctccagatgaaaatggtatgtttctaaccgaagaagagattgtagacaacatcttgttactactctttgcgggtcatgatacctcggctctttcaatcactttggtcatgaagactcttggcgaacattctgatgtttatgacaaggtgttaaaagagcaactagagatatcgaagacgaaagaagcatgggagtccctgaaatgggaggacatacaaaagatgaaatactcctggagtgttgtatgtgaagtcatgagactaaatccacctgttataggaacctatagagaggcccttgtggatattgattatgcgggttataccatcccgaaaggatggaagttacactggagtgctgtatcgacacaaagggacgaggctaactttgaagacgtaacacgttttgacccatcacggtttgaaggcgcaggaccgactccattcacctttgttccgtttggaggggggcctagaatgtgtttagggaaagaatttgctcgattggaagtacttgcgtttcttcacaatattgtcaccaatttcaaatgggacctgttgatacctgatgagaaaatagaatatgatcccatggctacccctgcaaaggggcttccaattcgtcttcatccccatcaagtttga

SEQ ID NO: 10 - protein, 476 aa, Stevia rebaudiana
MIQVLTPILLFLIFFVFWKVYKHQKTKINLPPGSFGWPFLGETLALLRAGWDSEPERFVRERIKKHGSPLVFKTSLFGDHFAVLCGPAGNKFLFCNENKLVASWWPVPVRKLFGKSLLTIRGDEAKWMRKMLLSYLGPDAFATHYAVTMDVVTRRHIDVHWRGKEEVNVFQTVKLYAFELACRLFMNLDDPNHIAKLGSLFNIFLKGIIELPIDVPGTRFYSSKKAGAAIRIELKKLIKARKLELKEGKASSSQDLLSHLLTSPDENGMFLTEEEIVDNILLLLFAGHDTSALSITLVMKTLGEHSDVYDKVLKEQLEISKTKEAWESLKWEDIQKMKYSWSVVCEVMRLNPPVIGTYREALVDIDYAGYTIPKGWKLHWSAVSTQRDEANFEDVTRFDPSRFEGAGPTPFTFVPFGGGPRMCLGKEFARLEVLAFLHNIVTNFKWDLLIPDEKIEYDPMATPAKGLPIRLHPHQV

SEQ ID NO: 11 - DNA, 1578 bp, Arabidopsis thaliana
atggagagtttggttgttcatacggtaaatgcaatttggtgcatagttattgtcggaatcttcagcgtaggttatcatgtgtatggaagagcggtggtggagcagtggaggatgcggaggagtttaaagttgcaaggcgtgaagggtcctccgccgtcgatctttaacggcaatgtgtcggagatgcaacggattcagtcggaggctaaacactgttccggcgataacatcatttctcatgactattcttcttctctatttcctcatttcgatcactggcgaaaacaatacggaaggatttacacatactcaacggggttaaagcagcacctttacataaaccacccggaaatggtgaaggagcttagccaaaccaacacacttaaccttggtagaatcactcacatcaccaaacgccttaaccccattctcggcaatggcatcatcacctctaatgggcctcattgggcccatcaacgtcgtatcattgcctatgagtttacccacgacaaaatcaagggaatggttggtttaatggtggaatctgccatgccaatgttgaacaaatgggaagagatggtgaaaagaggaggagaaatgggttgtgacataagagtggacgaagaccttaaggatgtctcagctgatgtcatcgctaaggcttgctttgggagctctttttcaaaaggcaaagcaatattctctatgattagggatcttttaaccgccattactaagcgaagcgtcctcttcagattcaatggcttcactgatatggtgtttggaagtaagaagcatggtgatgtggatattgatgcgcttgagatggaattagaatcttctatatgggaaacggttaaggagagggaaattgaatgtaaggatactcacaagaaggatctaatgcagttgatactcgagggagcgatgcgaagctgcgatggtaacttgtgggacaagtcagcctatagacggtttgtggtggacaattgcaagagcatctatttcgccggacatgattcaaccgcagtctcagtgtcttggtgccttatgctcctcgctctcaatcctagttggcaggttaaaattcgcgatgaaatcttgagttcttgcaagaatggcattcccgacgcagaatcaattcctaatctcaaaacggtgacaatggtaatacaagaaacaatgagactatacccaccagcaccaatcgtgggaagagaagcatccaaagacataagacttggagaccttgtggtgccaaaaggagtgtgcatttggacactcattcctgccttacaccgagaccccgagatctggggaccagacgcaaacgacttcaagccagagaggtttagtgagggaatctctaaggcttgcaaataccctcagtcatacatcccatttggccttggaccaagaacatgcgtaggcaaaaactttggtatgatggaagtgaaagtgcttgtttcacttattgtctcaaagttcagttttactctttccccgacttatcagcactctccaagccataaactccttgtagagcctcaacatggtgttgtcattagggttgtttga

SEQ ID NO: 12 - protein, 525 aa, Arabidopsis thaliana
MESLVVHTVNAIWCIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 13 - DNA, 1569 bp, Unknown
  Description of Unknown KAH2 polynucleotide
atgggtctcttccctttggaagatagttacacactcgtctttgaaggtttagcaataactctagctctctactacttattatccttcatctataaaacctctaaaaagacttgtactccacctaaagcaagcggtgagcaccctataacaggccacttaaaccttcttagtggttcatccggtcttccccatctagccttagcatctttggctgaccgatgtgggcccatattcaccgtccgacttggcatacgtagagttttggtggttagtaattgggaaattgctaaggagatcttcactacccatgatttgattgtttcaaaccgtcccaaatacctcgctgcaaagattttgggattcaactatgtgtccttttcgtttgctccatatggtccctattgggttggaatccgtaagatcatcgccacaaaactgatgtcaagtagcaggctccagaagcttcagtttgtccgagtttctgaactagaaaactccatgaaaagcatacgcgagtcttggaaagagaaaaaagacgaagaaggtaaagtgttggtggagatgaaaaaatggttttgggaattgaatatgaatatagttcttagaactgttgctggtaaacagtacactggaactgttgatgatgcggatgcgaagaggattagtgaattgtttagagaatggtttcattacacaggaaggtttgttgtgggagatgcttttccttttcttgggtggttggatttgggtggatataagaagaccatggaactagtggcttccagactagattccatggtctcaaaatggttagacgagcatcgcaaaaagcaggctaacgacgacaaaaaagaggacatggatttcatggacatcatgatatcgatgactgaagccaattcccctttggagggttatggtacggatacaataattaaaaccacttgcatgactcttattgtcagtggtgtagatacaacctccatcatgctaacttgggcactctcgttactactgaacaaccgtgacactcttaagaaagctcaagaagagctagacatgtgtgtgggaaaaggtcgacaagtaaacgaatcagatctagtaaacctaatctaccttgaagccgtattaaaagaagcattgcgactatacccagcagcattccttggaggtcctagagccttttcagaagactgcaccgtggcagggtaccgtatcccaaaaggcacatggctacttattaacatgtggaaacttcatcgtgatccaaacatatggtcagacccatgtgagtttaaaccagagaggttcttaaccccaaaccaaaaggacgtagatgttattggaatggattttgagttaatcccatttggtgcgggaagaaggtattgtccagggacacgtttggcattacaaatgttacacatagttctggccactctactacaaaactttgagatgtcaactccaaatgatgcacccgttgatatgaccgcgagtgttggaatgacaaatgcgaaggcaagtccacttgaagttctactttcgccacgtgttaagtggtcatag

SEQ ID NO: 14 - protein, 522 aa, Unknown
  Description of Unknown KAH2 polypeptide
MGLFPLEDSYTLVFEGLAITLALYYLLSFIYKTSKKTCTPPKASGEHPITGHLNLLSGSSGLPHLALASLADRCGPIFTVRLGIRRVLVVSNWEIAKEIFTTHDLIVSNRPKYLAAKILGFNYVSFSFAPYGPYWVGIRKIIATKLMSSSRLQKLQFVRVSELENSMKSIRESWKEKKDEEGKVLVEMKKWFWELNMNIVLRTVAGKQYTGTVDDADAKRISELFREWFHYTGRFVVGDAFPFLGWLDLGGYKKTMELVASRLDSMVSKWLDEHRKKQANDDKKEDMDFMDIMISMTEANSPLEGYGTDTIIKTTCMTLIVSGVDTTSIMLTWALSLLLNNRDTLKKAQEELDMCVGKGRQVNESDLVNLIYLEAVLKEALRLYPAAFLGGPRAFSEDCTVAGYRIPKGTWLLINMWKLHRDPNIWSDPCEFKPERFLTPNQKDVDVIGMDFELIPFGAGRRYCPGTRLALQMLHIVLATLLQNFEMSTPNDAPVDMTASVGMTNAKASPLEVLLSPRVKWS

SEQ ID NO: 15 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 16 - protein, 525 aa, Arabidopsis thaliana; identical to SEQ ID NO: 12

SEQ ID NO: 17 - protein, 522 aa, Unknown
  Description of Unknown Glycosyltransferase polypeptide
MGLFPLEDSYALVFEGLAITLALYYLLSFIYKTSKKTCTPPKASGEHPITGHLNLLSGSSGLPHLALASLADRCGPIFTIRLGIRRVLVVSNWEIAKEIFTTHDLIVSNRPKYLAAKILGFNYVSFSFAPYGPYWVGIRKIIATKLMSSSRLQKLQFVRVFELENSMKSIRESWKEKKDEEGKVLVEMKKWFWELNMNIVLRTVAGKQYTGTVDDADAKRISELFREWFHYTGRFVVGDAFPFLGWLDLGGYKKTMELVASRLDSMVSKWLDEHRKKQANDDKKEDMDFMDIMISMTEANSPLEGYGTDTIIKTTCMTLIVSGVDTTSIVLTWALSLLLNNRDTLKKAQEELDMCVGKGRQVNESDLVNLIYLEAVLKEALRLYPAAFLGGPRAFLEDCTVAGYRIPKGTCLLINMWKLHRDPNIWSDPCEFKPERFLTPNQKDVDVIGMDFELIPFGAGRRYCPGTRLALQMLHIVLATLLQNFEMSTPNDAPVDMTASVGMTNAKASPLEVLLSPRVKWS

(19) United States
(12) Patent Application Publication
(10) Pub. No.: US 2013/0338348 A1
(43) Pub. Date: Dec. 19, 2013

(54) STEVIOL AND STEVIOL GLYCOSIDE FORMATION IN PLANTS

(71) Applicant: J.R. Simplot Company, Boise, ID (US)

(72) Inventors: Caius M. ROMMENS, Boise, ID (US); Jingsong YE, Boise, ID (US); Roshani SHAKYA, Boise, ID (US)

(73) Assignee: J.R. Simplot Company, Boise, ID (US)

(21) Appl. No.: 13/826,505

(22) Filed: Mar. 14, 2013

Related U.S. Application Data
(60) Provisional application No. 61/618,079, filed on Mar. 30, 2012.

(51) Int. Cl. C12N 15/82; A23L 1/221

(52) U.S. Cl. CPC C12N 15/8245; A23L 1/221
USPC 536/18.1; 800/278; 800/298; 800/317.2; 435/320.1; 562/498

(57) ABSTRACT

Steviol glycosides are sweeter than sugar and have a much lower calorimetric value. The compounds are purified from leaves of Stevia and Rubus plants and used as sweetener in foods and beverages. The present methods use recombinant and genetic methods to produce steviol and steviol glycosides in plants and plant products.

### FIELD OF THE INVENTION

The field of this inventive technology concerns the genetic modification of the level of steviol and/or kaurenoic acid in a plant.

### BACKGROUND

Steviol glycosides are sweeter than sugar and have a much lower calorimetric value. The compounds are purified from leaves of Stevia and Rubus plants and used as sweetener in foods and beverages. There is broad interest in sweeter fruits and vegetables that are low in calorimetric value. The present inventive technology provides methods to produce steviol and steviol glycosides, as well as kaurenoic acid, the precursor of steviol, in plants.

### SUMMARY OF THE INVENTION

One aspect of the present invention method is based on the expression of, or the overexpression of, at least one of three different Stevia rebaudiana genes encoding 1-deoxy-D-xylulose 5-phosphate reductoisomerase (SrDxr), ent-copalyl diphosphate synthase (SrCps), and kaurenoic acid 13-hydroxylase (SrKah), respectively. Surprisingly, plants expressing these 3 genes produce steviol.

One aspect of the present invention is a method for producing steviol and/or kaurenoic acid, comprising expressing at least one of the DXR (1-deoxy-D-xylulose 5-phosphate reductoisomerase), CPPS (ent-copalyl diphosphate synthase or copalyl diphosphate synthase), and KAH (kaurenoic acid 13-hydroxylase) genes in a plant.

Another aspect of the present invention is method for producing steviol and/or kaurenoic acid, comprising transforming a plant with one or more expression cassettes that express at least one of the DXR, CPPS, and KAH genes, in the plant.

In one embodiment, the CPPS gene comprises either the DNA sequence of SEQ ID NO: 1, or encodes the protein of SEQ ID NO:2; wherein the DXR gene comprises either the DNA sequence of SEQ ID NO: 5, or encodes the protein of SEQ ID NO:6; and wherein the KAH gene comprises either the DNA sequence of SEQ ID NO: 9, or encodes the protein of SEQ ID NO:10.

Another aspect of the present invention is a method of altering the taste of a food obtained from a plant, comprising modifying the production of steviol in the plant. In one embodiment the production of steviol in the plant is increased. In another embodiment, the production of steviol in the plant is decreased. In one embodiment, the method of increasing the production of steviol in the plant comprises increasing the level of at least one of 2-C-methyl-D-erythitol-4 phosphate (MEP), and geranylgeranyl diphosphate (GGDP). In another embodiment, the modification of the production of steviol is achieved by expressing at least one of the DXR, CPPS, and KAH genes in the plant. In one embodiment, the taste of the food is sweeter than the taste of the same food obtained from a plant whose steviol production has not been modified as described herein. In one embodiment, the food comprises fruit. In another embodiment, the food is a fruit. In one embodiment, the fruit is selected from the group consisting of Apple, Apricot, Avocado, Banana, Bilberry, Blackberry, Blackcurrant, Blueberry, Currant, Cherry, Cherimoya, Clementine, Date, Damson, Dragonfruit, Durian, Eggplant, Elderberry, Feijoa, [[Gooseberry], Grape, Grapefruit, Guava, Huckleberry, Jackfruit, Jambul, Kiwi fruit, Kumquat, Legume, Lemon, Lime, Lychee, Mandarine, Mango, Mangostine, Melon, Cantaloupe, Honeydew melon, Watermelon, Rock melon, Nectarine, Orange, Peach, Pear, Williams pear or Bartlett pear, Pitaya, Physalis, Plum/prune (dried plum), Pineapple, Pomegranate, Raisin, Raspberry, Western raspberry (blackcap), Rambutan, Redcurrant, Salal berry, Satsuma, Star fruit, Strawberry, Tangerine, Tomato, Ugli fruit, Watermelon, and Ziziphus mauritiana. In one embodiment, the food comprises strawberries. In another embodiment, the food is a strawberry. In another embodiment, the food comprises a vegetable. In another embodiment, the food is a vegetable. In one embodiment, the vegetable is selected from the group consisting of Alfalfa sprouts, Anise, Artichoke, Arugula, Asparagus, Aubergine, Eggplant, Beans and peas, Azuki beans (or adzuki), Bean sprouts, Black beans, Black-eyed peas, Borlotti beans, Broad beans, Chickpeas, Garbanzos, or ceci beans, Green beans, Kidney beans, Lentils, Lima bean or Butter bean, Mung beans, Navy beans, Runner beans, Soy beans, Peas, Mangetout or Snap peas, Bok choy, Chinese leaves in the UK, Breadfruit, Broccoflower (a hybrid), Broccoli, Brussels sprouts, Cabbage, Calabrese, Cauliflower, Celery, Chard, Cilantro, Collard greens, Corn salad, Endive, Fennel, Fiddleheads (young coiled fern leaves), Frisee, Kale, Kohlrabi, Lemon grass, Lettuce Lactuca sativa, Maize, Corn, Sweetcorn, Mushrooms, Mustard greens, Nettles, New Zealand spinach, Okra, Onion family, Chives, Garlic, Leek Allium porrum, Onion, Shallot, Spring onion, Green onion, Scallion, Parsley, Peppers, Green pepper and Red pepper, bell pepper, pimento, Chili pepper, Capsicum, Jalapeno, Habanero, Paprika, Tabasco, Cayenne pepper, Radicchio, Rhubarb, Root vegetables, Beetroot, Beet, mangel-wurzel: a variety of beet used mostly as cattlefeed, Carrot, Celeriac, Daikon, Fennel, Radish, Swede, Rutabaga, Turnip, Wasabi, White radish, Salsify, Skirret, Spinach, Squashes, Acorn squash, Butternut squash, Courgette, Zucchini, Cucumber, Gem squash, Marrow, Squash, Cucurbita maxima, Patty pans, Pumpkin, Spaghetti squash, Tat soi, Tomato, Tubers, Jicama, Jerusalem artichoke, Potato, Sweet potato, Taro, Yam, Water chestnut, and Watercress.

### BRIEF DESCRIPTION OF THE DRAWINGS

FIG. 1. Map of pSIM1647

FIG. 2. RNA gel blot analysis of 1647 lines and controls.

FIG. 3A. LC-MS/MS data showing extracted ion chromatogram (EIC) of kaurenoic acid in Annona glabra, Stevia rebaudiana, and SrCPS potato Ranger Russet. K1, K2 and K3 represent kaurenoic acids produced in Annona glabra leaves (known to produce high levels of kaurenoic acid). K2 is also produced in Stevia rebaudiana. 401: transgenic control line carrying the T-DNA of pSIM401, which only contains the nptII selectable marker gene. Note that line 1647-17 contains detectable amounts of K2.

FIG. 3B. Mass spectra showing MS/MS fragmentation of K1, K2 and K3 kaurenoic acid of molecular mss m/z 301 in negative.

FIG. 4. Map of pSIM1651

FIG. 5. RNA gel blot analysis of 1651 (SrDxr) potatoes and untransformed controls.

FIG. 6. Map of pSIM1653

FIG. 7. SrDxs and SrDxr transcript levels in 1653 potato.

FIG. 8. Displays a western blot with geranylgeranyl diphosphate (GGPP) synthase antibodies, demonstrating high levels of SrDxr gene expression, but not SrDxs gene expression

FIG. 9. Map of pSIM1650

FIG. 10. LC/MS analysis of kaurenoic acid extracts from N. benthamiana agroinfiltrated with 1647 (SrCps) and from control N. benthamiana agroinfiltrated with 401.

FIG. 11. RNA gel blot analysis of N. benthamiana agroinfiltrated with 1647 (SrCps) and of control N. benthamiana agroinfiltrated with 401.

### DETAILED DESCRIPTION

There is little known about the genes required for the biosynthesis of steviol glycosides. A yeast strain overexpressing the Arabidopsis CYP714A2 cDNA (SEQ ID 15), designated tentatively as steviol synthase, appeared to convert some ent-kaurenoic acid to ent-7β,13-dihydroxykaerenoic acid (Yamaguchi et al., Method for producing steviol synthetase gene and steviol, US Patent application 2008/0271205A1). Recent studies, however, demonstrated that CYP714A2 is involved in gibberellin deactivation (Zhang et al., Plant J 67: 342-53, 2011). An alternative yeast strain overexpressing the Stevia P450 cDNA named 8-40 (SEQ ID 16), which encodes a protein that shares only very weak homology with the above-mentioned protein, also appeared to convert some kaurenoic acid into steviol (Brandle and Richman, Compositions and methods for producing steviol and steviol glycosides, U.S. Pat. No. 7,927,851).

Steviol is a diterpenoic compound with chemical name ent-kaur-16-en-13-ol-19-oic acid. Steviol is the aglycone of sweet glycosides accumulated in Stevia rebaudiana Bertoni. This compound is the hydroxylated form of ent-kaurenoic acid (ent-kaur-16-en-19-oic acid; ent-KA). Stevia leaf is used as a sweetening agent and contains several sweet glycosides. Indeed, stevia has been used for centuries as a natural sweetener. The plant contains sweet ent-kaurene glycosides with the most intense sweetness belonging to the species S. rebaudiana. Stevia has been evaluated for sweetness in animal response testing. In humans, stevia as a sweetening agent works well in weight-loss programs to satisfy sugar cravings and is low in calories, and the glycoside rebaudioside A is in commercially available products in the United States and has not shown any pharmacologic effects. Japan is the largest consumer of stevia leaves and uses the plant to sweeten foods, such as soy sauce, confections, and soft drinks, and as a replacement for aspartame and saccharin. Several studies have examined the pharmacologic effects of stevia in animals and humans. These studies were conducted on different stevia glycosides and contribute to the conflicting results. In addition, some of the earlier studies did not specify the glycoside content of the stevia used. Stevioside appears to have more pharmacologic effect than the commercially available sweeteners that primarily contain rebaudioside A. Stevia may be helpful in treating diabetes and hypertension.

The present inventors isolated genes from Stevia rebaudiana which are involved in the biosynthesis pathway for the production of steviol and/or kaurenoic acid. See Kumar et al., Gene, 492: 276-284 (2012), which is incorporated herein by reference. Thus, one aspect of the present invention method is based on the expression of, or the overexpression of, at least one of three different Stevia rebaudiana genes encoding 1-deoxy-D-xylulose 5-phosphate reductoisomerase (SrDxr), ent-copalyl diphosphate synthase (SrCps), and kaurenoic acid 13-hydroxylase (SrKah), respectively. Surprisingly, plants expressing these 3 genes produce steviol. Accordingly, one surprising application of the present invention comprises producing steviol and/or kaurenoic acid in a plant that does not normally produce steviol and/or kaurenoic acid or which produces steviol and/or kaurenoic acid in low levels. Thus, the present invention makes it possible to not only increase the levels of steviol and/or kaurenoic acid in plants that normally produce steviol and/or kaurenoic acid but to also create de novo one or more levels of steviol and/or kaurenoic acid in a plant not normally known to produce steviol and/or kaurenoic acid, such as potatoes. Table 1 herein provides data showing kaurenoic acid levels, the precursor of steviol, in potato lines transformed according to the present invention and Stevia rebaudiana thereby demonstrating that steviol levels can be increased in plants by genetically expressing one or more of the genes identified herein.

One aspect of the increase in steviol levels is to make the food sweeter than the same food obtained from a plant whose steviol level has not been modified. Thus, one aspect of the present invention is a method for producing steviol anchor kaurenoic acid, comprising expressing at least one of the DXR (1-deoxy-D-xylulose 5-phosphate reductoisomerase), CPPS (ent-copalyl diphosphate synthase or copalyl diphosphate synthase), and KAH (kaurenoic acid 13-hydroxylase) genes in a plant. Another aspect of the present invention is method for producing steviol and/or kaurenoic acid, comprising transforming a plant with one or more expression cassettes that express at least one of the DXR, CPPS, and KAH genes, in the plant. The Examples herein disclose how to make vectors and expression cassettes for expressing these genes. In one embodiment, the CPPS gene comprises either the DNA sequence of SEQ ID NO: 1, or encodes the protein of SEQ ID NO:2; wherein the DXR gene comprises either the DNA sequence of SEQ ID NO: 5, or encodes the protein of SEQ ID NO:6; and wherein the KAH gene comprises either the DNA sequence of SEQ ID NO: 9, or encodes the protein of SEQ ID NO:10.

Another aspect of the present invention is a method of altering the taste of a food obtained from a plant, comprising modifying the production of steviol in the plant. In one embodiment the production of steviol in the plant is increased. In another embodiment, the production of steviol in the plant is decreased. In one embodiment, the method of increasing the production of steviol in the plant comprises increasing the level of at least one of 2-C-methyl-D-erythitol-4 phosphate (MEP), and geranylgeranyl diphosphate (GGDP). In another embodiment, the modification of the production of steviol is achieved by expressing at least one of the DXR, CPPS, and KAH genes in the plant. In one embodiment, the taste of the food is sweeter than the taste of the same food obtained from a plant whose steviol production has not been modified as described herein. In one embodiment, the food comprises fruit. In another embodiment, the food is a fruit. In one embodiment, the fruit is selected from the group consisting of Apple, Apricot, Avocado, Banana, Bilberry, Blackberry, Blackcurrant, Blueberry, Currant, Cherry, Cherimoya, Clementine, Date, Damson, Dragonfruit, Durian, Eggplant, Elderberry, Feijoa, Gooseberry, Grape, Grapefruit, Guava, Huckleberry, Jackfruit, Jambul, Kiwi fruit, Kumquat, Legume, Lemon, Lime, Lychee, Mandarine, Mango, Mangostine, Melon, Cantaloupe, Honeydew melon, Watermelon, Rock melon, Nectarine, Orange, Peach, Pear, Williams pear or Bartlett pear, Pitaya, Physalis, Plum/prune (dried plum), Pineapple, Pomegranate, Raisin, Raspberry, Western raspberry (blackcap), Rambutan, Redcurrant, Salal berry, Satsuma, Star fruit, Strawberry, Tangerine, Tomato, Ugli fruit, Watermelon, and Ziziphus mauritiana. In one embodiment, the food comprises strawberries. In another embodiment, the food is a strawberry. In another embodiment, the food comprises a vegetable. In another embodiment, the food is a vegetable. In one embodiment, the vegetable is selected from the group consisting of Alfalfa sprouts, Anise, Artichoke, Arugula, Asparagus, Aubergine, Eggplant, Beans and peas, Azuki beans (or adzuki), Bean sprouts, Black beans, Black-eyed peas, Borlotti beans, Broad beans, Chickpeas, Garbanzos, or ceci beans, Green beans, Kidney beans, Lentils, Lima bean or Butter bean, Mung beans, Navy beans, Runner beans, Soy beans, Peas, Mangetout or Snap peas, Bok choy, Chinese leaves in the UK, Breadfruit, Broccoflower (a hybrid), Broccoli, Brussels sprouts, Cabbage, Calabrese, Cauliflower, Celery, Chard, Cilantro, Collard greens, Corn salad, Endive, Fennel, Fiddleheads (young coiled fern leaves), Frisee, Kale, Kohlrabi, Lemon grass, Lettuce Lactuca sativa, Maize, Corn, Sweetcorn, Mushrooms, Mustard greens, Nettles, New Zealand spinach, Okra, Onion family, Chives, Garlic, Leek Allium porrum, Onion, Shallot, Spring onion, Green onion, Scallion, Parsley, Peppers, Green pepper and Red pepper, bell pepper, pimento, Chili pepper, Capsicum, Jalapeno, Habanero, Paprika, Tabasco, Cayenne pepper, Radicchio, Rhubarb, Root vegetables, Beetroot, Beet, mangel-wurzel: a variety of beet used mostly as cattlefeed, Carrot, Celeriac, Daikon, Fennel, Radish, Swede, Rutabaga, Turnip, Wasabi, White radish, Salsify, Skirret, Spinach, Squashes, Acorn squash, Butternut squash, Courgette, Zucchini, Cucumber, Gem squash, Marrow, Squash, Cucurbita maxima, Patty pans, Pumpkin, Spaghetti squash, Tat soi, Tomato, Tubers, Jicama, Jerusalem artichoke, Potato, Sweet potato, Taro, Yam, Water chestnut, and Watercress.

### Method for Modifying a Plant

Many embodiments of the present invention relate to a method for modifying a plant, comprising expressing de novo or overexpressing at least one of the DXR gene, the CPPS gene, and the KAH gene, in the plant.

As described herein, “expressing de novo” means expressing a polypeptide that is not normally expressed in a plant, while “overexpressing” means expressing a polypeptide at a level higher than its normal expression level in a plant.

The de novo expression or overexpression of the CPPS gene can increase the production of, for example, kaurenoic acid, which can be converted to steviol and steviol glucoside. The CPPS gene can be cloned from, for example, a steviol or steviol glucoside producing plant such as Stevia rebaudiana, and optionally modified. The CPPS gene can either comprise the DNA sequence of SEQ ID NO: 1, or encode the protein of SEQ ID NO:2.

The de novo expression or overexpression of the DXR gene can up-regulate the expression of, for example, geranylgeranyl diphosphate synthase, which can increase the production of geranylgeranyl diphosphate, a precursor of kaurenoic acid. The DXR gene can be cloned from, for example, a steviol or steviol glucoside producing plant such as Stevia rebaudiana, and optionally modified. The DXR gene can either comprise the DNA sequence of SEQ ID NO: 5, or encode the protein of SEQ ID NO:6.

The de novo expression or overexpression of the KAH gene can increase the production of, for example, steviol from kaurenoic acid. The KAH gene can be cloned from, for example, a steviol or steviol glucoside producing plant such as Stevia rebaudiana, and optionally modified. The KAH gene can either comprise the DNA sequence of SEQ ID NO: 9, or encode the protein of SEQ ID NO:10. The KAH gene can either comprise the DNA sequence of SEQ ID NO: 11, or encode the protein of SEQ ID NO:12. The KAH gene can either comprise the DNA sequence of SEQ ID NO: 13, or encode the protein of SEQ ID NO:14.

The method described herein can significantly increase the production of kaurenoic acid by, for example, expressing de novo or overexpressing both the CPPS gene and the DXR gene in a plant. The method described herein can significantly increase the production of steviol by, for example, expressing de novo or overexpressing both the CPPS gene and the KAH gene in a plant. The method described herein can significantly increase the production of steviol by, for example, expressing de novo or overexpressing the CPPS gene, the DXR gene, and the KAH gene in a plant.

The method described herein can increase the level of kaurenoic acid production by, for example, at least 20%, or at least 50%, or at least 100%, or at least 200%, or at least 500%, or at least 1000%, compared to a wild plant of the same variety. The concentration of kaurenoic acid can be, for example, at least 5%, or at least 10%, or at least 20%, or at least 30%, or at least 40%, or at least 50% of the kaurenoic acid concentration in a wild plant of Stevia rebaudiana.

The method described herein can increase the level of steviol production by, for example, at least 20%, or at least 50%, or at least 100%, or at least 200%, or at least 500%, or at least 1000%, compared to a wild plant of the same variety. The concentration of steviol can be, for example, at least 5%, or at least 10%, or at least 20%, or at least 30%, or at least 40%, or at least 50%, of the steviol concentration in a wild plant of Stevia rebaudiana.

In some embodiments, the plant described herein is a dicotyledonous plant. In some embodiments, the plant is a fruit plant or a vegetable plant. In one particular embodiment, the plant is potato. In another particular embodiment, the plant is strawberry.

The method described herein for producing steviol and/or kaurenoic acid can be implemented by, for example, transforming a plant with one or more expression cassettes that express in the plant at least one of the DXR, CPPS, and KAH genes. The method can be implemented by, for example, (A) stably integrating into the genome of at least one plant cell one or more exogenous genetic cassettes selected from the group consisting of (i) a gene expression cassette for expressing DXR, (ii) a gene expression cassette for expressing CPPS, and (iii) a gene expression cassette for expressing KAH, and (B) regenerating the transformed plant cell into a plant. In a preferred embodiment, Agrobacterium-mediated transformation is used to produce the transformed plant cell.

The method described herein can further comprise expressing de novo or overexpressing at least one glycosyltransferase, which will increase the production of steviol glucoside (e.g., stevioside, rebaudioside A) from steviol. The glycosyltransferase can be selected from, for example, the protein of SEQ ID NO:15, the protein of SEQ ID NO:16, and the protein of SEQ ID NO:17.

The method described herein can comprise, for example, extracting steviol from the modified plant. The method can comprise, for example, extracting steviol glucoside from the modified plants. The method can comprise, for example, extracting kaurenoic acid from the modified plants. The method described herein can further comprise, for example, incorporating the modified plant or the steviol or steviol glucoside extracted therefrom into a food product or a nutritional composition

### Transformation Vectors

Many embodiments of the present invention also relate to one or more transformation vectors for transforming plant cells. The transformation vector can comprise, for example, one or more expression cassettes selected from the group consisting of (i) a gene expression cassette for expressing the CPPS gene, (ii) a gene expression cassette for expressing the DXR gene, and (iii) a gene expression cassette for expressing the KAH gene.

The transformation vector can be, for example, a binary vector suitable for Agrobacterium-mediated transformation. See, e.g., Komori et al., Plant Physiology 145:1155-1160 (2007) and Hellens et al., Trends in Plant Science 5 (10):446-451 (2000), incorporated herein by reference in their entireties. The binary vector can comprise, for example, a transfer DNA region delineated by two T-DNA border or plant-derived border-like sequences, wherein the expression cassettes described herein are located in the transfer DNA region. See USP 2012/0297500, incorporated herein by reference in its entirety.

Agrobacterium stains suitable for transforming binary vectors are known in the art and described in, for example, Lee et al., Plant Physiology 146:325-332 (2008), incorporated herein by reference in its entirety. In one particular embodiment, the Agrobacterium stain harboring the transformation vector is LBA4404. In another particular embodiment, the Agrobacterium stain harboring the transformation vector is AGL-1.

The transformation vector can comprise, for example, a gene expression cassette for expressing the CPPS gene. The expression cassette can comprise, from 5′ to 3′, (i) a promoter functional in a plant cell, operably linked to (ii) at least one copy the CPPS gene or fragment thereof, and (iii) a terminator functional in a plant cell.

The transformation vector can comprise, for example, a gene expression cassette for expressing the DXR gene. The expression cassette can comprise, from 5′ to 3′, (i) a promoter functional in a plant cell, operably linked to (ii) at least one copy the DXR gene or fragment thereof, and (iii) a terminator functional in a plant cell.

The transformation vector can comprise, for example, a gene expression cassette for expressing the KAH gene. The expression cassette can comprise, from 5′ to 3′, (i) a promoter functional in a plant cell, operably linked to (ii) at least one copy the KAH gene or fragment thereof, and (iii) a terminator functional in a plant cell.

The transformation vector can comprise, for example, two or more gene expression cassettes. The transformation vector can comprise, for example, a first gene expression cassette for expressing the CPPS gene, a second gene expression cassette for expressing the DXR gene, and a third gene expression cassette for expressing the KAH gene.

### Modified Plants

Many embodiments of the present invention also relate to a modified plant comprising in its genome one or more exogenous genetic cassettes selected from the group consisting of (i) a gene expression cassette for expressing DXR, (ii) a gene expression cassette for expressing CPPS, and (iii) a gene expression cassette for expressing KAH.

The modified plant described herein can comprise an inserted CPPS gene expression cassette and have, for example, increased production of kaurenoic acid, which can be converted to steviol and steviol glucoside. The CPPS gene can be cloned from, for example, a steviol or steviol glucoside producing plant such as Stevia rebaudiana, and optionally modified. The CPPS gene can either comprise the DNA sequence of SEQ ID NO: 1, or encode the protein of SEQ ID N0:2.

The modified plant described herein can comprise an inserted DXR gene expression cassette and have, for example, increased production of geranylgeranyl diphosphate synthase for producing geranylgeranyl diphosphate, a precursor of kaurenoic acid. The DXR gene can be cloned from, for example, a steviol or steviol glucoside producing plant such as Stevia rebaudiana, and optionally modified. The DXR gene can either comprise the DNA sequence of SEQ ID NO: 5, or encode the protein of SEQ ID NO:6.

The modified plant described herein can comprise an inserted KAH gene expression cassette and have, for example, increased production of steviol from kaurenoic acid. The KAH gene can be cloned from, for example, a steviol or steviol glucoside producing plant such as Stevia rebaudiana, and optionally modified. The KAH gene can either comprise the DNA sequence of SEQ ID NO: 9, or encode the protein of SEQ ID NO:10. The KAH gene can either comprise the DNA sequence of SEQ ID NO: 11, or encode the protein of SEQ ID NO:12. The KAH gene can either comprise the DNA sequence of SEQ ID NO: 13, or encode the protein of SEQ ID NO:14.

The modified plant described herein can comprise an inserted CPPS gene expression cassette and an inserted DXR gene expression cassette and have significantly increased production of kaurenoic acid. The modified plant described herein can comprise an inserted CPPS gene expression cassette and an inserted KAH gene expression cassette and have significantly increased production of steviol. The modified plant described herein can comprise an inserted CPPS gene expression cassette, an inserted KAH gene expression cassette and an inserted DXR gene expression cassette, and have significantly increased production of steviol.

The modified plant described herein can produce, for example, at least 20% more, or at least 50% more, or at least 100% more, or at least 200% more, or at least 500% more, or at least 1000% more kaurenoic acid than a wild plant of the same variety. The concentration of kaurenoic acid can be, for example, at least 5%, or at least 10%, or at least 20%, or at least 30%, or at least 40%, or at least 50% of the kaurenoic acid concentration in a wild plant of Stevia rebaudiana.

The modified plant described herein can produce, for example, at least 20% more, or at least 50% more, or at least 100% more, or at least 200% more, or at least 500% more, or at least 1000% more steviol than a wild plant of the same variety. The concentration of steviol can be, for example, at least 5%, or at least 10%, or at least 20%, or at least 30%, or at least 40%, or at least 50%, of the steviol concentration in a wild plant of Stevia rebaudiana.

The modified plant described herein can have, for example, altered taste. The modified plant can be, for example, sweeter than a wild plant of the same variety.

In some embodiments, the modified plant described herein is a dicotyledonous plant. In some embodiments, the modified plant is a fruit plant or a vegetable plant. In one particular embodiment, the modified plant is potato. In another particular embodiment, the modified plant is strawberry.

### Food Products

Further embodiments relate to food products and/or nutritional compositions produced from the modified plants described herein. The food product and/or nutritional compositions can be made from, for example, a fruit or a vegetable. Compare to food products made from a wild plant of the same variety, the food product described herein can have lower calorimetric value at the same sweetness level.

### Additional Embodiments

Embodiment 1. A method for modifying a plant, comprising expressing de novo or overexpressing at least one of 1-deoxy-D-xylulose 5-phosphate reductoisomerase (DXR), ent-copalyl diphosphate synthase (CPPS), and kaurenoic acid 13-hydroxylase (KAH), in said plant.

Embodiment 2. The method of Embodiment 1, comprising expressing de novo or overexpressing both CPPS and KAH in said plant.

Embodiment 3. The method of Embodiment 1, comprising expressing de novo or overexpressing both CPPS and DXR in said plant.

Embodiment 4. The method of Embodiment 1, comprising expressing de novo or overexpressing CPPS, DXR and KAH in said plant.

Embodiment 5. The method of any of Embodiment 1-4, comprising expressing de novo or overexpressing the CPPS gene of Stevia rebaudiana in a plant other than Stevia rebaudiana.

Embodiment 6. The method of any of Embodiments 1 and 3-5, comprising expressing de novo or overexpressing the DXR gene of Stevia rebaudiana in a plant other than Stevia rebaudiana.

Embodiment 7. The method of any of Embodiment 1-2 and 4-6, comprising expressing de novo or overexpressing the KAH gene of Stevia rebaudiana in a plant other than Stevia rebaudiana.

Embodiment 8. The method of any of Embodiment 1-7, wherein the CPPS gene either comprises the DNA sequence of SEQ ID NO: 1, or encodes the protein of SEQ ID NO:2; wherein the DXR gene either comprises the DNA sequence of SEQ ID NO: 5, or encodes the protein of SEQ ID NO:6; and wherein the KAH gene either comprises the DNA sequence of SEQ ID NO: 9, or encodes the protein of SEQ ID NO:10.

Embodiment 9. The method of any of Embodiment 1-8, comprising transforming a plant with one or more expression cassettes that express at least one of the DXR, CPPS, and KAH genes, in the plant.

Embodiment 10. The method of any of Embodiment 1-9, comprising stably integrating into the genome of at least one plant cell one or more exogenous genetic cassettes selected from the group consisting of (i) a gene expression cassette for expressing DXR, (ii) a gene expression cassette for expressing CPPS, and (iii) a gene expression cassette for expressing KAH

Embodiment 11. The method of any of Embodiment 1-10, further comprising overexpressing or expressing de novo at least one glycosyltransferases in said plant.

Embodiment 12. The method of any of Embodiment 1-11, wherein said plant produces at least 50% more, at least 100% more, or at least 200% more kaurenoic acid than a wild plant of the same variety.

Embodiment 13. The method of any of Embodiment 1-12, wherein the kaurenoic acid concentration in said plant is at least 10%, at least 20%, or at least 30% of the kaurenoic acid concentration in a wild plant of Stevia rebaudiana.

Embodiment 14. The method of any of Embodiment 1-13, wherein said plant produces at least 50% more, at least 100% more, or at least 200% more steviol than a wild plant of the same variety.

Embodiment 15. The method of any of Embodiment 1-14, wherein the steviol concentration in said plant is at least 10%, at least 20%, or at least 30% of the steviol concentration in a wild plant of Stevia rebaudiana.

Embodiment 16. The method of any of Embodiment 1-15, wherein said plant is potato or strawberry.

Embodiment 17. A modified plant made according to the method of any of Embodiments 1-16.

Embodiment 18. A modified plant comprising in its genome one or more exogenous genetic cassettes selected from the group consisting of (i) a gene expression cassette for expressing DXR, (ii) a gene expression cassette for expressing CPPS, and (iii) a gene expression cassette for expressing KAH.

Embodiment 19. The plant of Embodiment 18, comprising both the CPPS gene expression cassette and the KAH gene expression cassette.

Embodiment 20. The plant of Embodiment 18, comprising both the CPPS gene expression cassette and the DXR gene expression cassette.

Embodiment 21. The plant of Embodiment 18, comprising the CPPS gene expression cassette, the DXR gene expression cassette, and the KAH gene expression cassette.

Embodiment 22. The plant of any of Embodiment 18-21, wherein the CPPS gene, the DXR gene, and the KAH gene are cloned from Stevia rebaudiana and optionally modified.

Embodiment 23. The plant of any of Embodiment 18-22, wherein said plant is potato or strawberry.

Embodiment 24. The plant of any of Embodiment 18-23, wherein said plant produces at least 50% more, at least 100% more, or at least 200% more kaurenoic acid than a wild plant of the same variety.

Embodiment 25. The plant of any of Embodiment 18-24, wherein the kaurenoic acid concentration in said plant is at least 10%, at least 20%, or at least 30% of the kaurenoic acid concentration in a wild plant of Stevia rebaudiana.

Embodiment 26. The plant of any of Embodiment 18-25, wherein said plant produces at least 50% more, at least 100% more, or at least 200% more steviol than a wild plant of the same variety.

Embodiment 27. The plant of any of Embodiment 18-26, wherein the steviol concentration in said plant is at least 10%, at least 20%, or at least 30% of the steviol concentration in a wild plant of Stevia rebaudiana.

Embodiment 28. A food product or nutritional supplement produced from the plant of any of Embodiment 17-27.

Embodiment 29. A plant transformation vector, comprising one or more genetic cassettes selected from the group consisting of (i) a gene expression cassette for expressing DXR, (ii) a gene expression cassette for expressing CPPS, and (iii) a gene expression cassette for expressing KAH.

Embodiment 30. A method for up-regulating the expression of geranylgeranyl diphosphate synthase in a plant, comprising overexpressing or expressing de novo the DXR gene in said plant.

Embodiment 31. A method for producing kaurenoic acid in a plant, comprising overexpressing or expressing de novo the CPPS gene in said plant.

### EXAMPLES

### Example 1

### Method Development

Extraction and purification of Kaurenoic acid: Accurately weighted freeze dried plant sample of about 200 mg was extracted two times with 1.5 mL of hexane using sonicator, heat for 40 min. Mixed Supernatants of all tubes were dried and re-suspended in 100 μl of methanol. Then extract was purified on SPE cartridge by applying to a preconditioned solid phase extraction (SPE) column (Water's Sep-pak C18 cartridges, 3 cc, 200 mg). The column was washed with 5 mL 40% methanol and 3 mL 70% acetonitrile and eluted with 3 mL of 90% acetonitrile. Then the eluate was evaporated to 100 μl using a SpeedVac. Purified extracts are then ready for analysis by HPLC/MS.

HPLC-MS/MS analysis of Kaurenoic acid: Analyses were carried on Agilent's HPLC consisted of on-line degasser, quaternary pump, temperature controlled autosampler, variable length DAD and mass spectrometer for analysis. Chromatographic separation was achieved using analytical column Zorbax Eclipse XDB-C18 (4.6×150 mm, 5-Micron, Agilent, USA). Column temperature was 40° C. The mobile phase was isocratic 70% acetonitrile in water at a flow of 1 mL min⁻¹. The injection volume was 20 μl. Detection wave length was set at 210 nm.

LC-MS was conducted with an Agilent 1200 LC/MS 6320 Ion Trap. Experiments were carried out with an ESI ion source in negative ion mode, auto MSⁿ, The source was operated using 350° C. drying gas (N2) at 12 L min⁻¹, 55 psi nebulizer gas.

Extraction and purification of Steviol and steviol glycosides: About 400 mg freeze dried and powdered leaves were extracted two times with 1 mL of 60% MeOH using sonicator at ˜40° C. for 48 min. Supernatants were concentrated under vacuum. Then all four tubes were mixed and continued evaporation until about 100 μl left. Then concentrated extract was purified on SPE C-18 cartridge using vacuum manifold to speed up the process. SPE column used was strata C18-E (55 um, 70 A, 500 mg/3 mL) from Phenomenex. Preconditioned SPE column was loaded with extract, washed with 5 ml 40% methanol and eluted sequentially with 3 mL 70% methanol and 2 mL 90% acetonitrile. The eluent was evaporated under vacuum to 100 ul using a SpeedVac.

HPLC-MS/MS analysis of Steviol and Steviol glycosides: An Agilent 1200 HPLC system equipped with an on-line degasser, quaternary pump, thermostat, autosampler, column heater, and DAD and MS for sample analysis. Chromatography was carried out on Zorbax NH2 (4.6×250 mm, 5-Micron) analytical column (Agilent, USA). The elution was carried on by isocratic mobile phase 80:20 (acetonitrile pH 5: water, v/v). Column temperature was 40° C. The flow rate was set as 1 mL min⁻¹. The injection volume was 150. Detection wave length was at 210 nm.

Agilent's 1200 LC/MS 6320 Ion trap Instrument was operated with an ESI source in negative ion mode, auto MSⁿ, Mass acquisition was carried out in the scan range 100-1000 m/z. The source was operated using 350° C. drying gas (N2) at 12 L min−1, 55 psi nebulizer gas.

### Example 2

### Generation of SrCps-Expressing Potato Plants Producing the Steviol Precursor Kaurenoic Acid

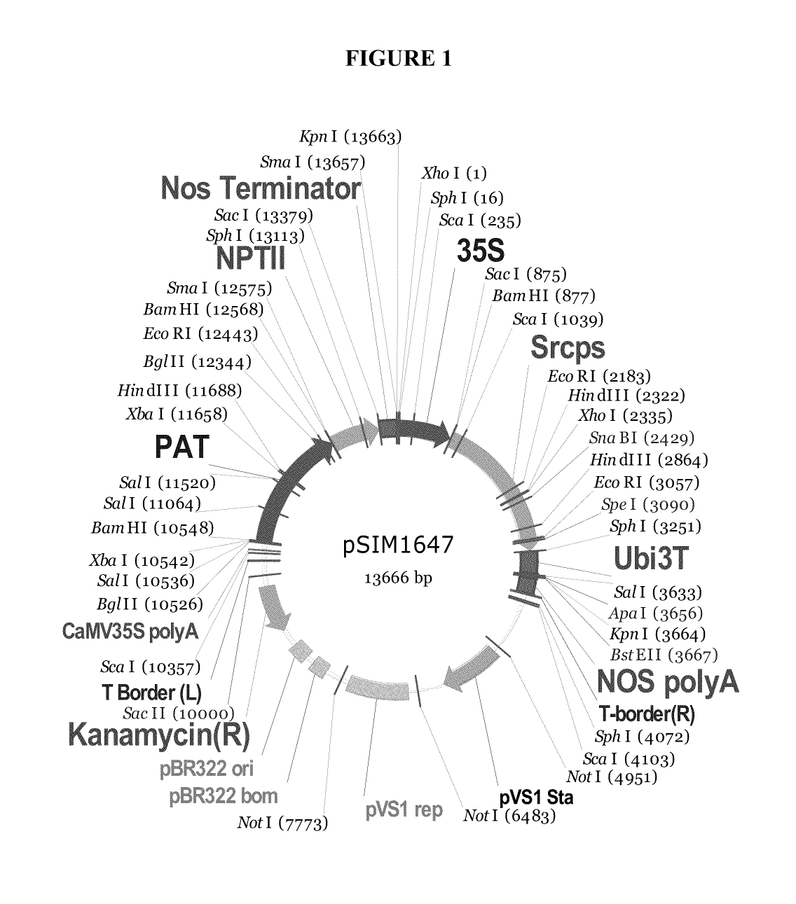

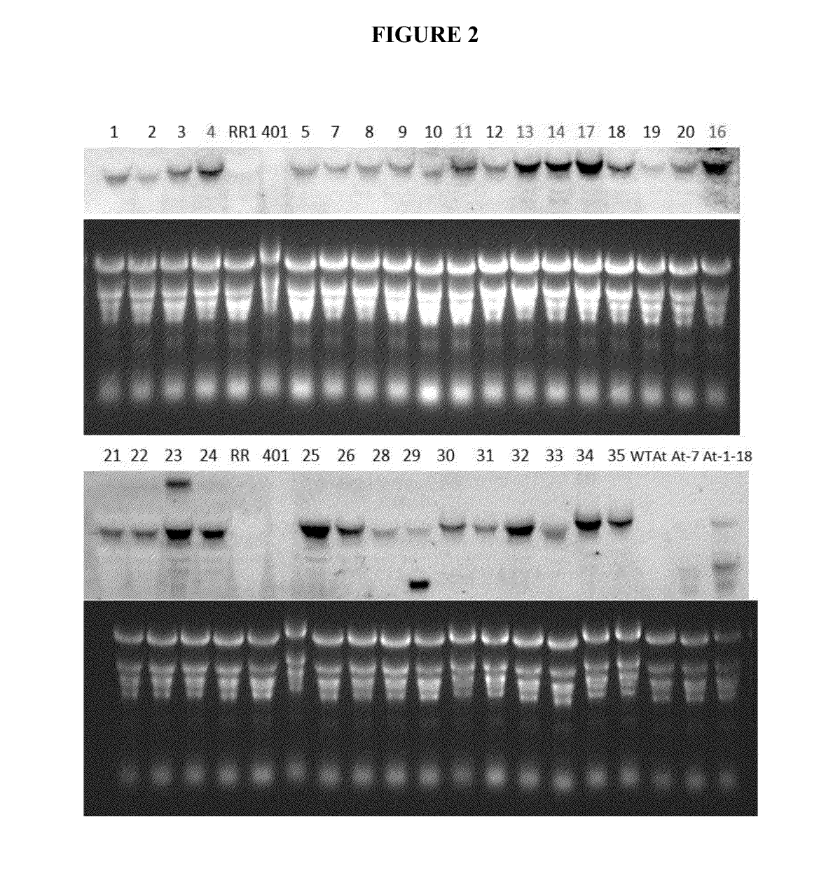

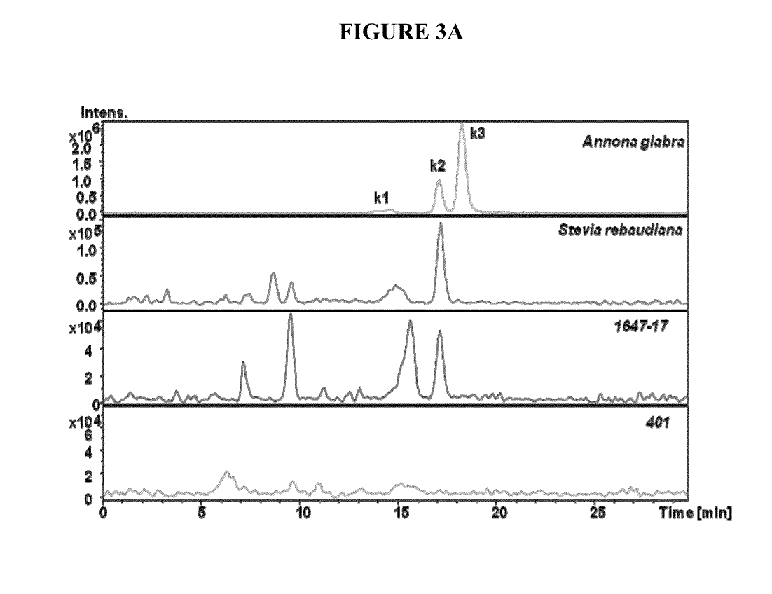

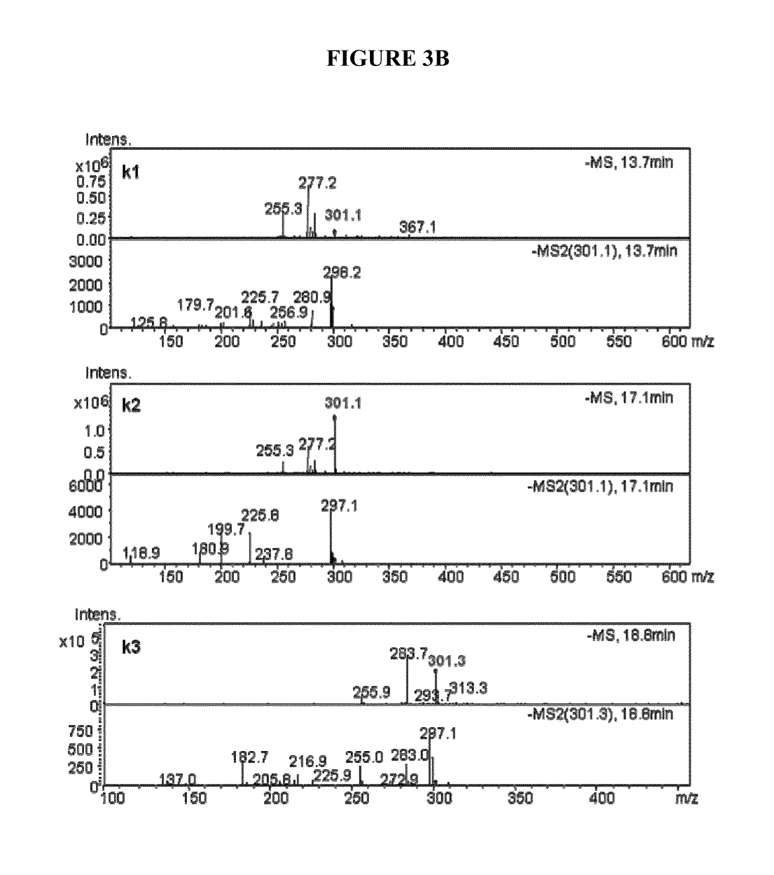

The SrCps cDNA (SEQ ID 1 for DNA, SEQ ID 2 for amino acid sequence) was operably linked to the 35S promoter of cauliflower mosaic virus (SEQ ID 3 for promoter) and the terminator of the potato ubiquitin 3 gene (SEQ ID 4 for terminator), and the expression cassette was inserted into a binary vector also containing the neomycin phosphotransferase (nptII) selectable marker gene. The resulting vector pSIM1647 (FIG. 1) was introduced into Agrobacterium strain LBA4404 as follows. Competent LB4404 cells (50 μL) were incubated for 5 minutes at 37° C. in the presence of 1 μg of vector DNA, frozen for about 15 seconds in liquid nitrogen (about −196° C.), and incubated again at 37° C. for 5 minutes. After adding 1 mL of liquid broth (LB), the treated cells were grown for 3 hours at 28° C. and plated on LB/agar containing streptomycin (100 mg/L) and kanamycin (50 mg/L). The vector DNAs were then isolated from overnight cultures of individual LBA4404 colonies and examined by restriction analysis to confirm the presence of intact plasmid DNA. For potato transformations, ten-fold dilutions of overnight-grown cultures were grown for 5-6 hours, precipitated for 15 minutes at 2,800 RPM, washed with MS liquid medium (Phytotechnology) supplemented with sucrose (3%, pH 5.7), and resuspended in the same medium to 0.2 OD/600 nm. The resuspended cells were mixed and used to infect 0.4-0.6 mm internodal segments of the potato variety “Ranger Russet”. Infected stems were incubated for two days on co-culture medium (1/10 MS salts, 3% sucrose, pH 5.7) containing 6 g/L agar at 22° C. in a Percival growth chamber (16 hrs light) and subsequently transferred to callus induction medium (CIM, MS medium supplemented with 3% sucrose 3, 2.5 mg/L of zeatin riboside, 0.1 mg/L of naphthalene acetic acid, and 6 g/L of agar) containing timentin (150 mg/L) and kanamycin (100 mg/L). After one month of culture on CIM, explants were transferred to shoot induction medium (SIM, MS medium supplemented with 3% sucrose, 2.5 mg/L of zeatin riboside, 0.3 mg/L of giberellic acid GA3, and 6 g/L of agar) containing timentin and kanamycin (150 and 100 mg/L respectively) until shoots arose. Shoots arising at the end of regeneration period were transferred to MS medium with 3% sucrose, 6 g/L of agar and timentin (150 mg/L). Transgenic plants were transferred to soil and placed in a growth chamber (11 hours light, 25° C.). They were then propagated to produce lines, and 3 copies of each line were planted in the greenhouse. Northern analysis of leaf RNA demonstrated that most 1647 lines expressed the transgene, especially lines 1647-17 and 25 (FIG. 2). These two lines also contained the largest amount of a kaurenoic acid compound labeled K2, as determined by LC/MS. Identification of k2 kaurenoic acid was confirmed by comparing retention time and MS/MS fragmentation of molecular ion m/z 301 and in negative ion mode. Similar mass fragmentation pattern was observed in all three kaurenoic acid. The relative levels of K2 in 1647-17 tubers were 0.76, which is almost half that of Stevia rebaudiana (1.8) (FIG. 3A, 3B and Table 1). Neither the transgenic potato lines nor Stevia rebaudiana contained another kaurenoic acid compound, K3, which is the predominant form in Annona glabra.

### Example 3

### Generation of Dxr-Expressing Potato Plants Producing the Kaurenoic Acid Precursor Geranylgeranyl Diphosphate (GGPP)

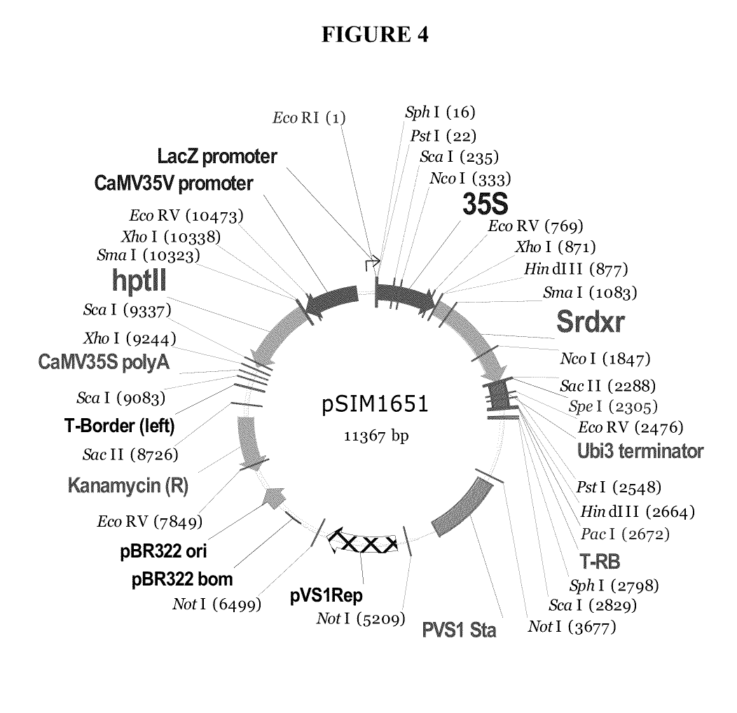

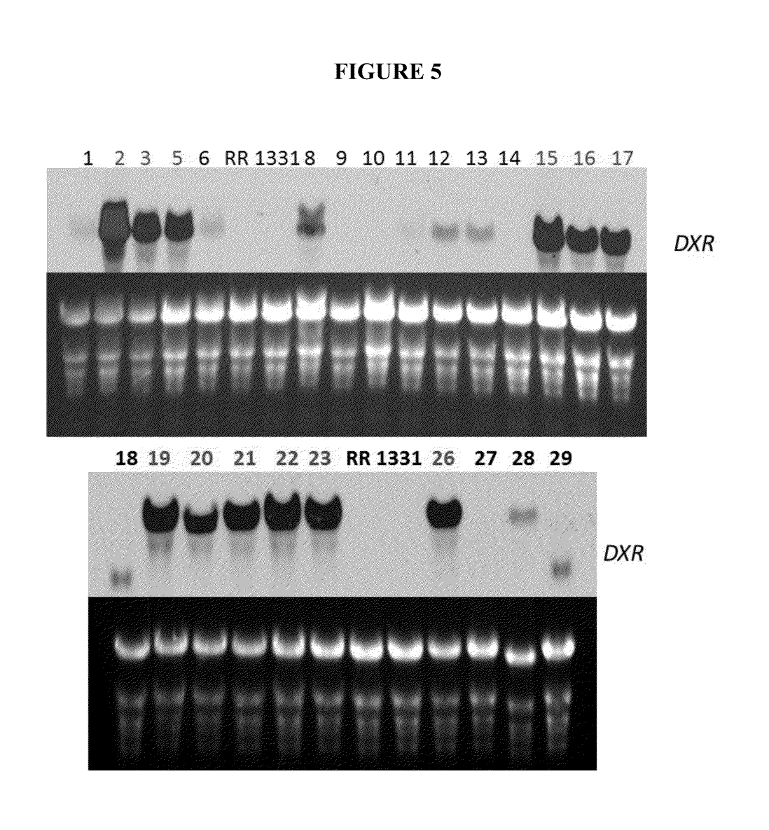

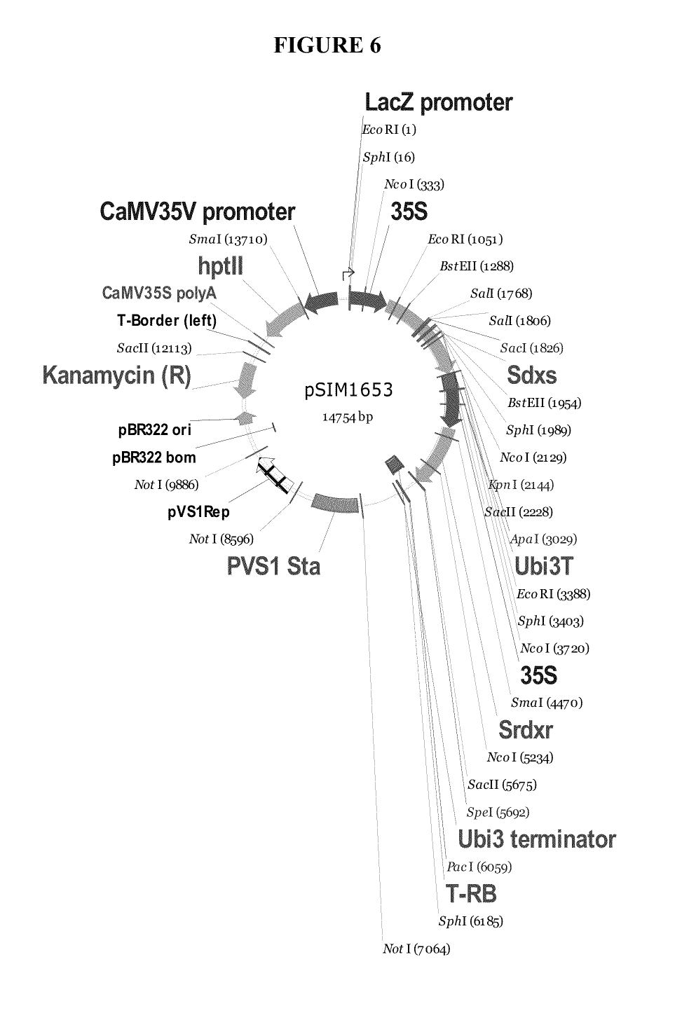

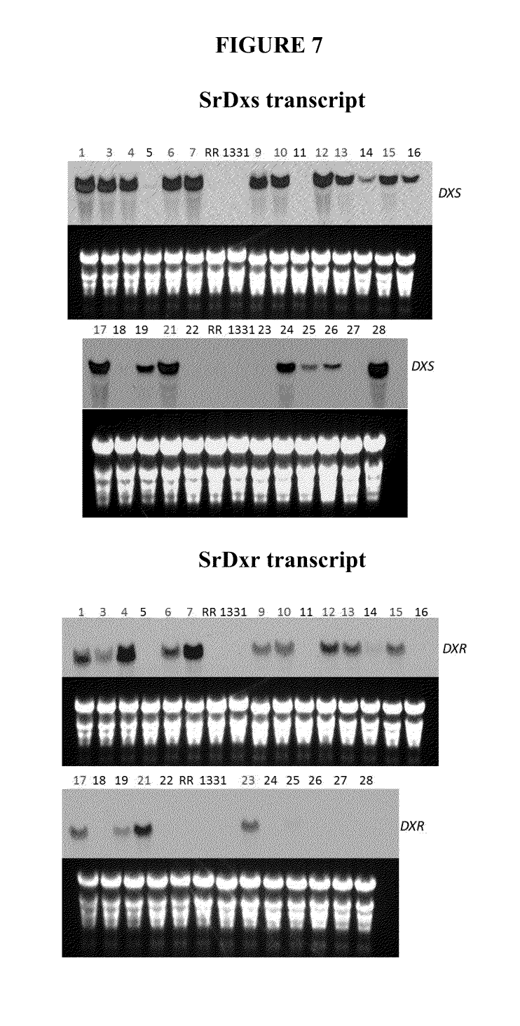

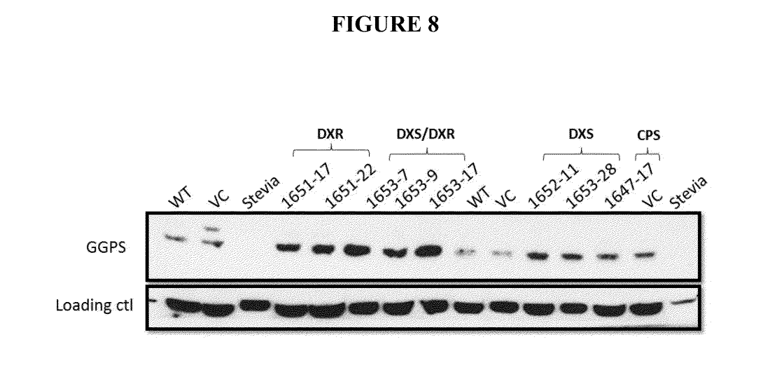

The binary vector pSIM1651 (FIG. 4) carries a cDNA of the Stevia rebaudiana Dxr gene (SEQ ID 5 for DNA, SEQ ID 6 for amino acid sequence) fused to the constitutive 35S promoter. The vector also contains the hygromycin phosphotransferase (hpt) gene as selectable marker for transformation. Transcript analysis of plants representing transgenic hygromycin resistant 1651 lines demonstrated that about half these lines expressed the transgene (FIG. 5). An additional vector, pSIM1652, was used to transform plants with a SrDxs gene (SEQ ID 7 for DNA, SEQ ID 8 for amino acid sequence) expression cassette, and plants were also transformed with a vector carrying expression cassettes for both SrDxr and SrDxs, named pSIM1653 (FIG. 6). See FIG. 7 for gene expression levels in 1653 plants. A western blot with geranylgeranyl diphosphate (GGPP) synthase antibodies demonstrated that high levels of SrDxr gene expression, but not SrDxs gene expression, were associated with about 4-8 fold increased amounts of this enzyme, which is involved in formation of the kaurenoic acid precursor GGPP (FIG. 8).

### Example 4

### Steviol Formation in Plants Expressing the SrCps, SrDxr, and SrKah Genes

The SrCps expressing line 1647-17 was retransformed with a construct carrying expression cassettes for cDNAs of both the SrDxr gene and the SrKah gene (see SEQ ID 9 for SrKah cDNA, SEQ ID 10 for amino acid sequence). Selectable markers were used to obtain doubly transformed plants. Another way to select for plants overexpressing SrDxr is by subjecting Agrobacterium-infected explants to fosmidomycin. Retransformed lines expressing all three transgenes are expected to produce greater amounts of kaurenoic acid than line 1647-17, and some of this kaurenoic acid is expected to be converted to steviol (See Kim, et al., Arch. Biochem. Biophys. 332 (2):223-230 (1996) and U.S. Pat. No. 7,927,851, both of which are incorporated herein by reference in their entireties).

Instead of the SrKah cDNA, it is possible to overexpress a Kah cDNA from Arabidopsis thaliana, shown in SEQ ID 11 for DNA, SEQ ID 12 for amino acid sequence.

### Example 5

### Stevioside Formation in Plants Also Expressing Glycosyltransferases

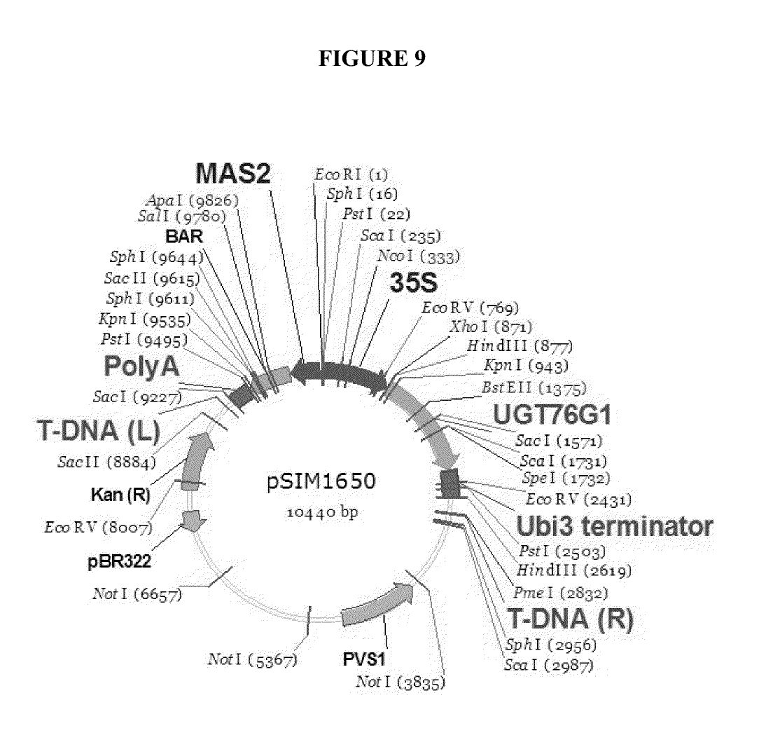

Plants can be retransformed using vectors carrying expression cassettes for specific glycosyltransferases that catalyze the transfer of sugar moieties from activated donor molecules to steviol or steviol-derivatives. Examples of such transferases are shown in SEQ IDs 15-17. One vector carrying a transferase is pSIM1650, shown in FIG. 9.

### Example 6

### Generation of SrCps-Transient Expressing Nicotiana benthamiana Producing the Steviol Precursor Kaurenoic Acid

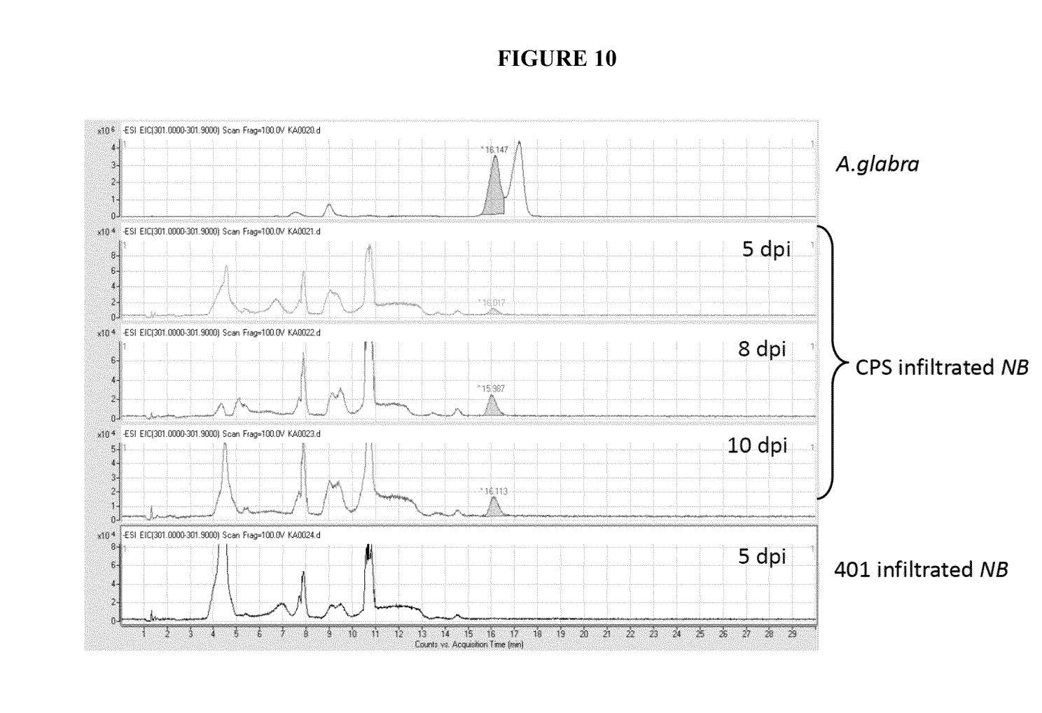

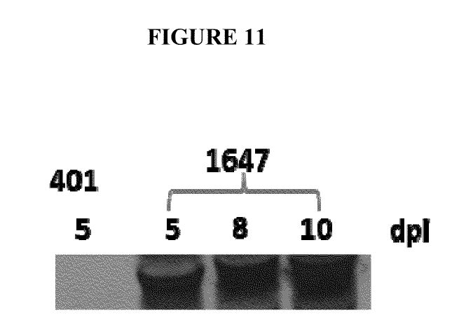

The binary vector pSIM1647 in Agrobacterium strain LBA4404 were used for transient expression of SrCps gene in N. benthamiana plants. Plants were grown in the greenhouse for 4-6 weeks (pre-flowering). For agroinfiltration, agrobacterium were grown overnight in shaker at 28° C. in 50 mL falcon tube with 10 mL of LB medium supplemented with streptomycin (100 mg/L) and kanamycin (50 mg/L). Optical density (OD) at 600 nm was measured on overnight culture. Agro culture was diluted in LB to bring OD₆₀₀ of 0.1-0.2. Cells were harvested by centrifugation for 10 min at 35000 rpm and resuspended into 1 mL infiltration buffer (10 mM MgCl2, 10 mM TrisHCl pH 7.5). OD was re-measured and diluted in infiltration buffer to make 0.25 OD₆₀₀. Then agroinfiltration was done into the underside of N. benthamiana leaves with 1 mL syringe. The youngest 3 leaves were used for best expression. After 8 days of infiltration, leaves were collected and immediately freeze in liquid N2. Kaurenoic acid was extracted from freeze dried leaves. LC/MS analysis of these leaf extract demonstrated that N. benthamiana produced kaurenoic acid, precursor of steviol (FIG. 10). Northern analysis determined the transient expression of 1647 (SrCps) transgene (FIG. 11).

SEQUENCES
SEQ IDs

SEQ ID 1 (CPS DNA)
ATGAAGACCGGCTTCATCTCTCCCGCCACCGTCTTCCACCACCGTATTTCTCCGGCAACCACCTTCCGCCACCACCT
TTCTCCGGCGACCACCAACTCCACTGGAATTGTAGCTCTTAGAGACATCAACTTCCGGTGTAAAGCGGTATCCAAAG
AGTACTCTGATTTACTACAAAAAGATGAGGCTTCATTTACCAAGTGGGACGATGACAAAGTGAAGGACCATTTGGAC
ACAAATAAGAATTTGTATCCAAACGATGAGATCAAGGAGTTTGTTGAGAGCGTGAAAGCAATGTTTGGTTCTATGAA
TGACGGAGAAATAAATGTGTCAGCGTATGATACGGCTTGGGTTGCACTCGTGCAAGATGTTGATGGAAGTGGTTCCC
CTCAATTTCCATCAAGTTTGGAGTGGATCGCGAACAATCAACTCTCAGATGGGTCTTGGGGCGATCATTTGTTATTT
TCGGCTCATGATAGGATCATTAACACGTTGGCATGTGTTATAGCGCTTACTTCTTGGAACGTCCATCCAAGTAAATG
TGAAAAAGGACTGAATTTTCTTAGAGAAAACATATGTAAACTCGAAGACGAGAACGCGGAACATATGCCAATTGGTT
TTGAAGTCACGTTCCCGTCGCTAATAGATATCGCAAAGAAGCTAAATATTGAAGTTCCTGAGGATACTCCTGCCTTA
AAAGAAATTTATGCAAGAAGAGACATAAAACTCACAAAGATACCAATGGAAGTATTGCACAAAGTGCCCACAACTTT
ACTTCATAGTTTGGAAGGAATGCCAGATTTGGAATGGGAAAAACTTCTGAAATTGCAATGCAAAGATGGATCATTTC
TGTTTTCTCCATCATCTACTGCTTTTGCACTCATGCAAACAAAAGATGAAAAGTGTCTTCAGTATTTGACAAATATT
GTTACCAAATTCAATGGTGGAGTTCCGAATGTGTACCCGGTGGATCTATTCGAACATATTTGGGTAGTTGATCGACT
TCAACGACTTGGGATTGCTCGTTATTTCAAATCAGAGATCAAAGATTGCGTTGAATATATTAACAAGTATTGGACAA
AGAATGGGATTTGTTGGGCAAGAAACACGCACGTACAAGATATTGATGATACCGCAATGGGATTTAGGGTTTTAAGA
GCACATGGTTATGATGTTACTCCAGATGTATTTCGACAATTTGAGAAGGATGGTAAATTCGTATGTTTCGCTGGACA
GTCAACACAAGCCGTCACCGGAATGTTCAATGTGTATAGAGCGTCACAAATGCTCTTTCCCGGAGAAAGAATTCTTG
AAGATGCAAAGAAATTTTCATATAATTATTTGAAAGAAAAACAATCGACAAATGAGCTTCTTGATAAATGGATCATC
GCCAAAGACTTACCTGGAGAGGTTGGATATGCGCTAGACATACCATGGTATGCAAGCTTACCGCGACTCGAGACAAG
ATATTACTTAGAGCAATACGGGGGCGAGGATGATGTTTGGATTGGAAAAACTCTATACAGGATGGGATATGTGAGCA
ATAATACGTACCTTGAAATGGCCAAATTGGACTACAATAACTATGTGGCCGTGCTTCAACTCGAATGGTACACTATC
CAGCAATGGTATGTTGATATCGGTATCGAAAAGTTTGAAAGTGACAATATCAAAAGCGTATTAGTGTCGTATTACTT
GGCTGCAGCCAGCATATTCGAGCCGGAAAGGTCCAAGGAACGAATCGCGTGGGCTAAAACCACCATATTAGTTGACA
AGATCACCTCAATTTTTGATTCATCACAATCCTCAAAAGAGGACATAACAGCCTTTATAGACAAATTTAGGAACAAA
TCGTCTTCTAAGAAGCATTCAATAAATGGAGAACCATGGCACGAGGTGATGGTTGCACTGAAAAAGACCCTACACGG
CTTCGCTTTGGATGCACTCATGACTCATAGTCAAGACATCCACCCGCAACTCCATCAAGCTTGGGAGATGTGGTTGA
CGAAATTGCAAGATGGAGTAGATGTGACAGCGGAATTAATGGTACAAATGATAAATATGACAGCTGGTCGTTGGGTA
TCCAAAGAACTTTTAACTCATCCTCAATACCAACGCCTCTCAACCGTCACAAATAGTGTGTGTCACGATATAACTAA
GCTCCATAACTTCAAGGAGAATTCCACGACGGTAGACTCGAAAGTTCAAGAACTAGTGCAACTTGTGTTTAGCGACA
CGCCCGATGATCTTGATCAGGATATGAAACAGACGTTTCTAACCGTCATGAAAACCTTCTACTACAAGGCGTGGTGT
GATCCGAACACGATAAATGACCATATCTCCAAGGTGTTCGAGATTGTAATATGA
SEQ ID 2 (CPS Protein)
MKTGFISPATVFHHRISPATTFRHHLSPATTNSTGIVALRDINFRCKAVSKEYSDLLQKDEASFTKWDDDKVKDHLD
TNKNLYPNDEIKEFVESVKAMFGSMNDGEINVSAYDTAWVALVQDVDGSGSPQFPSSLEWIANNQLSDGSWGDHLLF
SAHDRIINTLACVIALTSWNVHPSKCEKGLNFLRENICKLEDENAEHMPIGFEVTFPSLIDIAKKLNIEVPEDTPAL
KEIYARRDIKLTKIPMEVLHKVPTTLLHSLEGMPDLEWEKLLKLQCKDGSFLFSPSSTAFALMQTKDEKCLQYLTNI
VTKFNGGVPNVYPVDLFEHIWVVDRLQRLGIARYFKSEIKDCVEYINKYWTKNGICWARNTHVQDIDDTAMGFRVLR
AHGYDVTPDVFRQFEKDGKFVCFAGQSTQAVTGMFNVYRASQMLFPGERILEDAKKFSYNYLKEKQSTNELLDKWII
AKDLPGEVGYALDIPWYASLPRLETRYYLEQYGGEDDVWIGKTLYRMGYVSNNTYLEMAKLDYNNYVAVLQLEWYTI
QQWYVDIGIEKFESDNIKSVLVSYYLAAASIFEPERSKERIAWAKTTILVDKITSIFDSSQSSKEDITAFIDKFRNK
SSSKKHSINGEPWHEVMVALKKTLHGFALDALMTHSQDIHPQLHQAWEMWLTKLQDGVDVTAELMVQMINMTAGRWV
SKELLTHPQYQRLSTVTNSVCHDITKLHNFKENSTTVDSKVQELVQLVFSDTPDDLDQDMKQTFLTVMKTFYYKAWC
DPNTINDHISKVFEIVI
SEQ ID 3 (35S)
AGATTAGCCTTTTCAATTTCAGAAAGAATGCTAACCCACAGATGGTTAGAGAGGCTTACGCAGCAGGTCTCATCAAG
ACGATCTACCCGAGCAATAATCTCCAGGAAATCAAATACCTTCCCAAGAAGGTTAAAGATGCAGTCAAAAGATTCAG
GACTAACTGCATCAAGAACACAGAGAAAGATATATTTCTCAAGATCAGAAGTACTATTCCAGTATGGACGATTCAAG
GCTTGCTTCACAAACCAAGGCAAGTAATAGAGATTGGAGTCTCTAAAAAGGTAGTTCCCACTGAATCAAAGGCCATG
GAGTCAAAGATTCAAATAGAGGACCTAACAGAACTCGCCGTAAAGACTGGCGAACAGTTCATACAGAGTCTCTTACG
ACTCAATGACAAGAAGAAAATCTTCGTCAACATGGTGGAGCACGACACACTTGTCTACTCCAAAAATATCAAAGATA
CAGTCTCAGAAGACCAAAGGGCAATTGAGACTTTTCAACAAAGGGTAATATCCGGAAACCTCCTCGGATTCCATTGC
CCAGCTATCTGTCACTTTATTGTGAAGATAGTGGAAAAGGAAGGTGGCTCCTACAAATGCCATCATTGCGATAAAGG
AAAGGCCATCGTTGAAGATGCCTCTGCCGACAGTGGTCCCAAAGATGGACCCCCACCCACGAGGAGCATCGTGGAAA
AAGAAGACGTTCCAACCACGTCTTCAAAGCAAGTGGATTGATGTGATATCTCCACTGACGTAAGGGATGACGCACAA
TCCCACTATCCTTCGCAAGACCCTTCCTCTATATAAGGAAGTTCATTTCATTTGGAGAGAACACGGGGGAC
SEQ ID 4 (Ubi3T)
TTTTAATGTTTAGCAAATGTCCTATCAGTTTTCTCTTTTTGTCGAACGGTAATTTAGAGTTTTTTTTGCTATATGGA
TTTTCGTTTTTGATGTATGTGACAACCCTCGGGATTGTTGATTTATTTCAAAACTAAGAGTTTTTGCTTATTGTTCT
CGTCTATTTTGGATATCAATCTTAGTTTTATATCTTTTCTAGTTCTCTACGTGTTAAATGTTCAACACACTAGCAAT
TTGGCTGCAGCGTATGGATTATGGAACTATCAAGTCTGTGGGATCGATAAATATGCTTCTCAGGAATTTGAGATTTT
ACAGTCTTTATGCTCATTGGGTTGAGTATAATATAGTAAAAAAATAGG
SEQ ID 5 (DXR DNA)
ATGTCTTTGAGCTATCTATCTCCAACACAAACCAATCTAATCACTTTCTCCGACACCTGCAAATCCCAAACCCACCT
TCTCAAGCTCCAAGGTGGGTTTTGCTTCAAGAGAAAAGATGTTAAGCTCGCAGGAAAAGGGATTCGATGTTCGGCGC
AGCCTCCGCCGCCGCCGGCGTGGCCGGGAACGGCGCTGGTTGACCCCGGGACGAAGAATTGGGACGGCCCTAAACCT
ATTTCAATAGTGGGATCTACTGGTTCAATTGGGACTCAGACACTTGATATTGTTGCTGAAAACCCTGATAAGTTTCG
AGTTGTAGCACTTGCTGCTGGATCAAACGTGACTCTTCTTGCTGAACAGATAAAGGCATTCAAACCACAATTAGTTT
CAATCCAGAACGAATCTTTAGTTGGCGAACTTAAAGAAGCATTAGCTGATGCTGATTACATGCCTGAAATTATTCCC
GGAGATCAAGGCATCATTGAGGTCGCTCGCCATCCCGATTGTGTCACTGTTGTCACAGGCATAGTTGGTTGTGCTGG
TTTGAAGCCTACAGTTGCTGCCATTGAAGCAGGGAAAAACATAGCATTAGCTAATAAAGAAACCCTAATTGCCGGTG
GTCCGTTCGTTCTTCCTCTTGCACGTAAACATAATGTTAAAATTCTTCCTGCTGATTCAGAACATTCTGCTATATTC
CAGTGTATTCAAGGCTTTCCTGAAGGTGCTTTGAGGCGTATAATCTTAACCGCATCTGGTGGTGCTTTTAGAGATTT
ACCAGTTGAAAAACTAAAAGATGTTAAAGTAGCCGATGCATTAAAACATCCAAACTGGAGTATGGGTAAAAAAATCA
CGGTTGATTCAGCGACACTTTTCAACAAGGGTCTTGAAGTTATCGAAGCTCATTATCTTTACGGGTCAGATTATGAT
AATATTGAAATTGTTATTCATCCTCAATCTATCATACACTCCATGGTTGAGACACAGGACTCTTCGGTTCTAGCCCA
ATTAGGTTGGCCCGATATGCGTTTGCCAATTCTTTACACGTTATCTTGGCCCGATAGAATATCATGTTCTGAAATTA
CTTGGCCTCGCCTCGATCTTTGCAAGTTGGGATCATTAACATTTAAAGCTCCCGATAATGTGAAATACCCGTCGATG
GATTTGGCTTATGCCGCTGGACGAGCTGGCGGCACGATGACCGGAGTTCTTAGTGCCGCCAATGAGAAAGCGGTTGA
GATGTTCATTGATGAAAAGATTCAATATTTGGACATATTTAAAGTTGTTGAGCTAACATGTGCGAAACATCAATCCG
AACTCGTAACTGCACCGTCACTTGAAGAAATCGTGCATTATGACTTGTGGGCTCGTGATTATGCGGCTAGTTTGAAG
TCATCACCCGGTTTGACCGCGGTAGCTCTTGTATGA
SEQ ID 6 (DXR Protein)
MSLSYLSPTQTNLITFSDTCKSQTHLLKLQGGFCFKRKDVKLAGKGIRCSAQPPPPPAWPGTALVDPGTKNWDGPKP
ISIVGSTGSIGTQTLDIVAENPDKFRVVALAAGSNVTLLAEQIKAFKPQLVSIQNESLVGELKEALADADYMPEIIP
GDQGIIEVARHPDCVTVVTGIVGCAGLKPTVAAIEAGKNIALANKETLIAGGPFVLPLARKHNVKILPADSEHSAIF
QCIQGFPEGALRRIILTASGGAFRDLPVEKLKDVKVADALKHPNWSMGKKITVDSATLFNKGLEVIEAHYLYGSDYD
NIEIVIHPQSIIHSMVETQDSSVLAQLGWPDMRLPILYTLSWPDRISCSEITWPRLDLCKLGSLTFKAPDNVKYPSM
DLAYAAGRAGGTMTGVLSAANEKAVEMFIDEKIQYLDIFKVVELTCAKHQSELVTAPSLEEIVHYDLWARDYAASLK
SSPGLTAVALV
SEQ ID 7 (DXS DNA)
ATGGCGGTGGCAGGATCGACCATGAACCTGCATCTCACTTCATCTCCATACAAGACAGTTCCATCACTCTGTAAATT
CACCAGAAAACAGTTCCGATTAAAGGCCTCTGCAACGAATCCAGACGCTGAAGATGGGAAGATGATGTTTAAAAACG
ATAAACCCAATTTGAAGGTCGAATTCACTGGGGAGAAACCGGTGACACCATTACTGGATACCATTAATTACCCTGTG
CACATGAAAAACCTCACCACTCAGGATCTTGAGCAATTAGCAGCAGAACTTAGACAAGATATTGTATATTCAGTAGC
GAATACAGGTGGTCATTTGAGTTCAAGTTTAGGTGTTGTTGAATTGTCTGTTGCTTTACACCATGTTTTCAACACCC
CAGATGACAAGATCATTTGGGACGTTGGTCACCAGGCATACCCACATAAGATTTTGACCGGAAGAAGGTCAAAGATG
CACACCATAAGAAAAACTTCTGGTTTAGCTGGTTTTCCTAAACGAGATGAAAGTGCTCATGATGCTTTTGGTGCTGG
ACATAGTTCTACAAGCATCTCTGCTGGCCTAGGTATGGCTGTCGGTAGAGATTTATTAGGGAAAACCAACAACGTGA
TATCGGTGATCGGAGATGGCGCCATGACGGCCGGACAAGCATATGAGGCGATGAATAATGCAGGATTTCTTGATTCA
AATCTAATCGTCGTTTTAAACGACAACAAGCAAGTTTCATTACCGACTGCCACGTTGGACGGACCTGCAACTCCCGT
CGGGGCTCTCAGCGGCGCTTTATCCAAATTGCAAGCCAGTACCAAGTTCCGGAAGCTTCGTGAAGCCGCCAAGAGCA
TTACTAAACAAATTGGACCTCAAGCACATGAAGTGGCGGCGAAAGTCGACGAATACGCAAGAGGTATGATTAGTGCT
AGCGGGTCGACTTTATTCGAGGAGCTCGGATTATACTACATCGGTCCCGTCGATGGTCACAATGTTGAAGATTTAGT
CAACATTTTTGAAAAAGTCAAGTCAATGCCCGCACCCGGACCGGTTCTAATCCACATCGTGACCGAAAAAGGCAAAG
GTTACCCTCCTGCTGAAGCCGCTGCTGACCGCATGCACGGAGTTGTGAAGTTTGATGTTCCAACTGGAAAACAATTC
AAGACAAAATCACCGACACTTTCGTATACTCAGTATTTTGCTGAATCACTTATAAAAGAAGCTGAAGCTGATAACAA
GATTGTCGCGATACACGCCGCCATGGGAGGCGGTACCGGACTCAATTACTTCCAGAAGAAGTTTCCGGAACGTTGTT
TTGACGTCGGTATCGCGGAACAACACGCAGTTACTTTCGCCGCGGGTTTAGCCACCGAAGGTCTTAAACCATTTTGC
GCGATCTATTCGTCGTTTTTGCAACGAGGATACGATCAAGTGGTGCATGATGTTGATCTACAAAAGTTACCGGTTCG
GTTTGCGATGGACCGAGCTGGTTTAGTCGGGGCTGATGGACCGACACATTGTGGTGCGTTTGACATAACCTACATGG
CGTGTCTACCAAACATGGTGGTGATGGCTCCAGCCGATGAAGCCGAATTGATGCACATGGTTGCAACGGCTGCAGCC
ATTGACGACAGACCGAGTTGCTTTCGGTTCCCAAGAGGCAATGGCATTGGTGCACCACTTCCTCCTAATAACAAAGG
GATTCCCATAGAGGTTGGTAAAGGAAGAATATTACTTGAAGGAACTCGAGTTGCGATATTGGGATACGGTTCGATAG
TTCAAGAATGTCTAGGTGCGGCTAGCTTGCTTCAAGCCCATAACGTGTCTGCAACCGTAGCCGATGCGCGGTTCTGC
AAACCGTTAGACACCGGACTGATTAGACGATTAGCCAACGAGCATGAAGTCTTACTTACCGTAGAGGAAGGCTCGAT
TGGTGGATTTGGATCACACGTTGCTCACTTTCTAAGCTTAAATGGTCTCTTAGATGGAAAACTTAAGCTTAGAGCAA
TGACTCTTCCTGATAAATACATTGATCATGGTGCACCACAAGATCAGCTTGAAGAAGCCGGTCTTTCTTCAAAACAT
ATTTGTTCATCTCTTTTATCACTTTTGGGAAAACCTAAAGAAGCACTTCAATACAAATCAATAATGTAA
SEQ ID 8 (DXS Protein)
Sequence to be provided by Jingsong
MAVAGSTMNLHLTSSPYKTVPSLCKFTRKQFRLKASATNPDAEDGKMMFKNDKPNLKVEFTGEKPVTPLLDTINYPV
HMKNLTTQDLEQLAAELRQDIVYSVANTGGHLSSSLGVVELSVALHHVFNTPDDKIIWDVGHQAYPHKILTGRRSKM
HTIRKTSGLAGFPKRDESAHDAFGAGHSSTSISAGLGMAVGRDLLGKTNNVISVIGDGAMTAGQAYEAMNNAGFLDS
NLIVVLNDNKQVSLPTATLDGPATPVGALSGALSKLQASTKFRKLREAAKSITKQIGPQAHEVAAKVDEYARGMISA
SGSTLFEELGLYYIGPVDGHNVEDLVNIFEKVKSMPAPGPVLIHIVTEKGKGYPPAEAAADRMHGVVKFDVPTGKQF
KTKSPTLSYTQYFAESLIKEAEADNKIVAIHAAMGGGTGLNYFQKKFPERCFDVGIAEQHAVTFAAGLATEGLKPFC
AIYSSFLQRGYDQVVHDVDLQKLPVRFAMDRAGLVGADGPTHCGAFDITYMACLPNMVVMAPADEAELMHMVATAAA
IDDRPSCFRFPRGNGIGAPLPPNNKGIPIEVGKGRILLEGTRVAILGYGSIVQECLGAASLLQAHNVSATVADARFC
KPLDTGLIRRLANEHEVLLTVEEGSIGGFGSHVAHFLSLNGLLDGKLKLRAMTLPDKYIDHGAPQDQLEEAGLSSKH
ICSSLLSLLGKPKEALQYKSIM
SEQ ID 9 (KAH DNA)
ATGATTCAAGTTCTAACACCGATCCTCCTCTTCCTCATTTTCTTCGTTTTCTGGAAGGTTTACAAGCACCAGAAAAC
CAAAATCAATCTTCCACCGGGAAGCTTCGGATGGCCATTTCTGGGCGAAACTCTGGCACTTCTACGTGCAGGTTGGG
ATTCAGAGCCGGAGAGATTTGTTCGTGAACGGATCAAGAAACACGGAAGTCCTCTAGTGTTTAAGACGTCGTTGTTT
GGCGACCATTTTGCGGTGTTGTGTGGACCTGCCGGAAACAAGTTCCTGTTCTGCAACGAGAACAAGCTGGTGGCGTC
GTGGTGGCCGGTTCCGGTGAGGAAGCTTTTCGGCAAGTCTCTGCTCACGATTCGTGGTGATGAAGCTAAGTGGATGA
GGAAGATGTTGTTATCGTATCTTGGTCCTGATGCTTTCGCAACTCATTATGCCGTCACAATGGATGTCGTCACCCGT
CGGCATATCGACGTTCATTGGCGAGGGAAAGAAGAGGTGAACGTATTCCAAACCGTTAAGTTATATGCCTTTGAGCT
TGCATGTCGTTTATTCATGAACCTAGACGACCCAAACCACATTGCAAAACTCGGTTCCTTGTTCAACATTTTTTTGA
AAGGCATCATTGAGCTTCCAATCGACGTCCCAGGGACACGATTTTATAGCTCCAAAAAAGCAGGAGCAGCTATCAGG
ATTGAACTAAAAAAATTGATTAAAGCAAGAAAACTGGAACTGAAAGAAGGGAAGGCATCATCTTCACAAGACCTCTT
ATCACATTTGCTTACATCTCCAGATGAAAATGGTATGTTTCTAACCGAAGAAGAGATTGTAGACAACATCTTGTTAC
TACTCTTTGCGGGTCATGATACCTCGGCTCTTTCAATCACTTTGGTCATGAAGACTCTTGGCGAACATTCTGATGTT
TATGACAAGGTGTTAAAAGAGCAACTAGAGATATCGAAGACGAAAGAAGCATGGGAGTCCCTGAAATGGGAGGACAT
ACAAAAGATGAAATACTCCTGGAGTGTTGTATGTGAAGTCATGAGACTAAATCCACCTGTTATAGGAACCTATAGAG
AGGCCCTTGTGGATATTGATTATGCGGGTTATACCATCCCGAAAGGATGGAAGTTACACTGGAGTGCTGTATCGACA
CAAAGGGACGAGGCTAACTTTGAAGACGTAACACGTTTTGACCCATCACGGTTTGAAGGCGCAGGACCGACTCCATT
CACCTTTGTTCCGTTTGGAGGGGGGCCTAGAATGTGTTTAGGGAAAGAATTTGCTCGATTGGAAGTACTTGCGTTTC
TTCACAATATTGTCACCAATTTCAAATGGGACCTGTTGATACCTGATGAGAAAATAGAATATGATCCCATGGCTACC
CCTGCAAAGGGGCTTCCAATTCGTCTTCATCCCCATCAAGTTTGA
SEQ ID 10 (KAH Protein)
MIQVLTPILLFLIFFVFWKVYKHQKTKINLPPGSFGWPFLGETLALLRAGWDSEPERFVRERIKKHGSPLVFKTSLF
GDHFAVLCGPAGNKFLFCNENKLVASWWPVPVRKLFGKSLLTIRGDEAKWMRKMLLSYLGPDAFATHYAVTMDVVTR
RHIDVHWRGKEEVNVFQTVKLYAFELACRLFMNLDDPNHIAKLGSLFNIFLKGIIELPIDVPGTRFYSSKKAGAAIR
IELKKLIKARKLELKEGKASSSQDLLSHLLTSPDENGMFLTEEEIVDNILLLLFAGHDTSALSITLVMKTLGEHSDV
YDKVLKEQLEISKTKEAWESLKWEDIQKMKYSWSVVCEVMRLNPPVIGTYREALVDIDYAGYTIPKGWKLHWSAVST
QRDEANFEDVTRFDPSRFEGAGPTPFTFVPFGGGPRMCLGKEFARLEVLAFLHNIVTNFKWDLLIPDEKIEYDPMAT
PAKGLPIRLHPHQV
SEQ ID 11 (AtKAH DNA)
ATGGAGAGTTTGGTTGTTCATACGGTAAATGCAATTTGGTGCATAGTTATTGTCGGAATCTTCAGCGTAGGTTATCA
TGTGTATGGAAGAGCGGTGGTGGAGCAGTGGAGGATGCGGAGGAGTTTAAAGTTGCAAGGCGTGAAGGGTCCTCCGC
CGTCGATCTTTAACGGCAATGTGTCGGAGATGCAACGGATTCAGTCGGAGGCTAAACACTGTTCCGGCGATAACATC
ATTTCTCATGACTATTCTTCTTCTCTATTTCCTCATTTCGATCACTGGCGAAAACAATACGGAAGGATTTACACATA
CTCAACGGGGTTAAAGCAGCACCTTTACATAAACCACCCGGAAATGGTGAAGGAGCTTAGCCAAACCAACACACTTA
ACCTTGGTAGAATCACTCACATCACCAAACGCCTTAACCCCATTCTCGGCAATGGCATCATCACCTCTAATGGGCCT
CATTGGGCCCATCAACGTCGTATCATTGCCTATGAGTTTACCCACGACAAAATCAAGGGAATGGTTGGTTTAATGGT
GGAATCTGCCATGCCAATGTTGAACAAATGGGAAGAGATGGTGAAAAGAGGAGGAGAAATGGGTTGTGACATAAGAG
TGGACGAAGACCTTAAGGATGTCTCAGCTGATGTCATCGCTAAGGCTTGCTTTGGGAGCTCTTTTTCAAAAGGCAAA
GCAATATTCTCTATGATTAGGGATCTTTTAACCGCCATTACTAAGCGAAGCGTCCTCTTCAGATTCAATGGCTTCAC
TGATATGGTGTTTGGAAGTAAGAAGCATGGTGATGTGGATATTGATGCGCTTGAGATGGAATTAGAATCTTCTATAT
GGGAAACGGTTAAGGAGAGGGAAATTGAATGTAAGGATACTCACAAGAAGGATCTAATGCAGTTGATACTCGAGGGA
GCGATGCGAAGCTGCGATGGTAACTTGTGGGACAAGTCAGCCTATAGACGGTTTGTGGTGGACAATTGCAAGAGCAT
CTATTTCGCCGGACATGATTCAACCGCAGTCTCAGTGTCTTGGTGCCTTATGCTCCTCGCTCTCAATCCTAGTTGGC
AGGTTAAAATTCGCGATGAAATCTTGAGTTCTTGCAAGAATGGCATTCCCGACGCAGAATCAATTCCTAATCTCAAA
ACGGTGACAATGGTAATACAAGAAACAATGAGACTATACCCACCAGCACCAATCGTGGGAAGAGAAGCATCCAAAGA
CATAAGACTTGGAGACCTTGTGGTGCCAAAAGGAGTGTGCATTTGGACACTCATTCCTGCCTTACACCGAGACCCCG
AGATCTGGGGACCAGACGCAAACGACTTCAAGCCAGAGAGGTTTAGTGAGGGAATCTCTAAGGCTTGCAAATACCCT
CAGTCATACATCCCATTTGGCCTTGGACCAAGAACATGCGTAGGCAAAAACTTTGGTATGATGGAAGTGAAAGTGCT
TGTTTCACTTATTGTCTCAAAGTTCAGTTTTACTCTTTCCCCGACTTATCAGCACTCTCCAAGCCATAAACTCCTTG
TAGAGCCTCAACATGGTGTTGTCATTAGGGTTGTTTGA
SEQ ID 12 (AtKAH Protein)
MESLVVHTVNAIWCIVIVGIFSVGYHVYGRAVVEQWRMRRSLKLQGVKGPPPSIFNGNVSEMQRIQSEAKHCSGDNI
ISHDYSSSLFPHFDHWRKQYGRIYTYSTGLKQHLYINHPEMVKELSQTNTLNLGRITHITKRLNPILGNGIITSNGP
HWAHQRRIIAYEFTHDKIKGMVGLMVESAMPMLNKWEEMVKRGGEMGCDIRVDEDLKDVSADVIAKACFGSSFSKGK
AIFSMIRDLLTAITKRSVLFRFNGFTDMVFGSKKHGDVDIDALEMELESSIWETVKEREIECKDTHKKDLMQLILEG
AMRSCDGNLWDKSAYRRFVVDNCKSIYFAGHDSTAVSVSWCLMLLALNPSWQVKIRDEILSSCKNGIPDAESIPNLK
TVTMVIQETMRLYPPAPIVGREASKDIRLGDLVVPKGVCIWTLIPALHRDPEIWGPDANDFKPERFSEGISKACKYP
QSYIPFGLGPRTCVGKNFGMMEVKVLVSLIVSKFSFTLSPTYQHSPSHKLLVEPQHGVVIRVV
SEQ ID 13 (Kah2 DNA)
ATGGGTCTCTTCCCTTTGGAAGATAGTTACACACTCGTCTTTGAAGGTTTAGCAATAACTCTAGCTCTCTACTACTT
ATTATCCTTCATCTATAAAACCTCTAAAAAGACTTGTACTCCACCTAAAGCAAGCGGTGAGCACCCTATAACAGGCC
ACTTAAACCTTCTTAGTGGTTCATCCGGTCTTCCCCATCTAGCCTTAGCATCTTTGGCTGACCGATGTGGGCCCATA
TTCACCGTCCGACTTGGCATACGTAGAGTTTTGGTGGTTAGTAATTGGGAAATTGCTAAGGAGATCTTCACTACCCA
TGATTTGATTGTTTCAAACCGTCCCAAATACCTCGCTGCAAAGATTTTGGGATTCAACTATGTGTCCTTTTCGTTTG
CTCCATATGGTCCCTATTGGGTTGGAATCCGTAAGATCATCGCCACAAAACTGATGTCAAGTAGCAGGCTCCAGAAG
CTTCAGTTTGTCCGAGTTTCTGAACTAGAAAACTCCATGAAAAGCATACGCGAGTCTTGGAAAGAGAAAAAAGACGA
AGAAGGTAAAGTGTTGGTGGAGATGAAAAAATGGTTTTGGGAATTGAATATGAATATAGTTCTTAGAACTGTTGCTG
GTAAACAGTACACTGGAACTGTTGATGATGCGGATGCGAAGAGGATTAGTGAATTGTTTAGAGAATGGTTTCATTAC
ACAGGAAGGTTTGTTGTGGGAGATGCTTTTCCTTTTCTTGGGTGGTTGGATTTGGGTGGATATAAGAAGACCATGGA
ACTAGTGGCTTCCAGACTAGATTCCATGGTCTCAAAATGGTTAGACGAGCATCGCAAAAAGCAGGCTAACGACGACA
AAAAAGAGGACATGGATTTCATGGACATCATGATATCGATGACTGAAGCCAATTCCCCTTTGGAGGGTTATGGTACG
GATACAATAATTAAAACCACTTGCATGACTCTTATTGTCAGTGGTGTAGATACAACCTCCATCATGCTAACTTGGGC
ACTCTCGTTACTACTGAACAACCGTGACACTCTTAAGAAAGCTCAAGAAGAGCTAGACATGTGTGTGGGAAAAGGTC
GACAAGTAAACGAATCAGATCTAGTAAACCTAATCTACCTTGAAGCCGTATTAAAAGAAGCATTGCGACTATACCCA
GCAGCATTCCTTGGAGGTCCTAGAGCCTTTTCAGAAGACTGCACCGTGGCAGGGTACCGTATCCCAAAAGGCACATG
GCTACTTATTAACATGTGGAAACTTCATCGTGATCCAAACATATGGTCAGACCCATGTGAGTTTAAACCAGAGAGGT
TCTTAACCCCAAACCAAAAGGACGTAGATGTTATTGGAATGGATTTTGAGTTAATCCCATTTGGTGCGGGAAGAAGG
TATTGTCCAGGGACACGTTTGGCATTACAAATGTTACACATAGTTCTGGCCACTCTACTACAAAACTTTGAGATGTC
AACTCCAAATGATGCACCCGTTGATATGACCGCGAGTGTTGGAATGACAAATGCGAAGGCAAGTCCACTTGAAGTTC
TACTTTCGCCACGTGTTAAGTGGTCATAG
>SEQ ID 14 (Kah2 protein)
MGLFPLEDSYTLVFEGLAITLALYYLLSFIYKTSKKTCTPPKASGEHPITGHLNLLSGSSGLPHLALASLADRCGPI
FTVRLGIRRVLVVSNWEIAKEIFTTHDLIVSNRPKYLAAKILGFNYVSFSFAPYGPYWVGIRKIIATKLMSSSRLQK
LQFVRVSELENSMKSIRESWKEKKDEEGKVLVEMKKWFWELNMNIVLRTVAGKQYTGTVDDADAKRISELFREWFHY
TGRFVVGDAFPFLGWLDLGGYKKTMELVASRLDSMVSKWLDEHRKKQANDDKKEDMDFMDIMISMTEANSPLEGYGT
DTIIKTTCMTLIVSGVDTTSIMLTWALSLLLNNRDTLKKAQEELDMCVGKGRQVNESDLVNLIYLEAVLKEALRLYP
AAFLGGPRAFSEDCTVAGYRIPKGTWLLINMWKLHRDPNIWSDPCEFKPERFLTPNQKDVDVIGMDFELIPFGAGRR
YCPGTRLALQMLHIVLATLLQNFEMSTPNDAPVDMTASVGMTNAKASPLEVLLSPRVKWS
SEQ ID 15 (UGT76G1 Protein)
MENKTETTVRRRRRIILFPVPFQGHINPILQLANVLYSKGFSITIFHTNFNKPKTSNYPHFTFRFILDNDPQDERIS
NLPTHGPLAGMRIPIINEHGADELRRELELLMLASEEDEEVSCLITDALWYFAQSVADSLNLRRLVLMTSSLFNFHA
HVSLPQFDELGYLDPDDKTRLEEQASGFPMLKVKDIKSAYSNWQILKEILGKMIKQTKASSGVIWNSFKELEESELE
TVIREIPAPSFLIPLPKHLTASSSSLLDHDRTVFQWLDQQPPSSVLYVSFGSTSEVDEKDFLEIARGLVDSKQSFLW
VVRPGFVKGSTWVEPLPDGFLGERGRIVKWVPQQEVLAHGAIGAFWTHSGWNSTLESVCEGVPMIFSDFGLDQPLNA
RYMSDVLKVGVYLENGWERGEIANAIRRVMVDEEGEYIRQNARVLKQKADVSLMKGGSSYESLESLVSYISSL
SEQ ID 16 (CYP714A2 Protein)
MESLVVHTVNAIWCIVIVGIFSVGYHVYGRAVVEQWRMRRSLKLQGVKGPPPSIFNGNVSEMQRIQSEAKHCSGDNI
ISHDYSSSLFPHFDHWRKQYGRIYTYSTGLKQHLYINHPEMVKELSQTNTLNLGRITHITKRLNPILGNGIITSNGP
HWAHQRRIIAYEFTHDKIKGMVGLMVESAMPMLNKWEEMVKRGGEMGCDIRVDEDLKDVSADVIAKACFGSSFSKGK
AIFSMIRDLLTAITKRSVLFRFNGFTDMVFGSKKHGDVDIDALEMELESSIWETVKEREIECKDTHKKDLMQLILEG
AMRSCDGNLWDKSAYRRFVVDNCKSIYFAGHDSTAVSVSWCLMLLALNPSWQVKIRDEILSSCKNGIPDAESIPNLK
TVTMVIQETMRLYPPAPIVGREASKDIRLGDLVVPKGVCIWTLIPALHRDPEIWGPDANDFKPERFSEGISKACKYP
QSYIPFGLGPRTCVGKNFGMMEVKVLVSLIVSKFSFTLSPTYQHSPSHKLLVEPQHGVVIRVV
SEQ ID 17 (8-40)
MGLFPLEDSYALVFEGLAITLALYYLLSFIYKTSKKTCTPPKASGEHPITGHLNLLSGSSGLPHLALASLADRCGPI
FTIRLGIRRVLVVSNWEIAKEIFTTHDLIVSNRPKYLAAKILGFNYVSFSFAPYGPYWVGIRKIIATKLMSSSRLQK
LQFVRVFELENSMKSIRESWKEKKDEEGKVLVEMKKWFWELNMNIVLRTVAGKQYTGTVDDADAKRISELFREWFHY
TGRFVVGDAFPFLGWLDLGGYKKTMELVASRLDSMVSKWLDEHRKKQANDDKKEDMDFMDIMISMTEANSPLEGYGT
DTIIKTTCMTLIVSGVDTTSIVLTWALSLLLNNRDTLKKAQEELDMCVGKGRQVNESDLVNLIYLEAVLKEALRLYP
AAFLGGPRAFLEDCTVAGYRIPKGTCLLINMWKLHRDPNIWSDPCEFKPERFLTPNQKDVDVIGMDFELIPFGAGRR
YCPGTRLALQMLHIVLATLLQNFEMSTPNDAPVDMTASVGMTNAKASPLEVLLSPRVKWS

### Tables

TABLE 1
Kaurenoic acid levels (based on MS peak area) in potato and Stevia
rebaudiana.

| | Kaurenoic acid | | | | Lines for |
|---|---|---|---|---|---|
| | K1 or K1- | | | Northern | retransformation |
| Lines | like | K2 | K3 | data | w/DXS/DXR |
| 1647-4 | 0.88 | 0.38 | no | ++ | No |
| 1647-13 | 1.1 | 0.64 | no | +++ | No |
| 1647-17 | 0.87 | 0.76 | no | ++++ | Yes |
| 1647-23 | 0.84 | 0.38 | no | ++++ | No |
| 1647-24 | 1.3 | 0.43 | no | +++ | No |
| 1647-25 | 0.78 | 0.67 | no | ++++ | No |
| 1647-26 | 0.83 | 0.32 | no | ++ | No |
| 1647-32 | 1.1 | 0.5 | no | +++ | No |
| 1647-34 | 1.1 | 0.56 | no | +++ | No |
| RR wt 1 | 1.8 | no | no | Faint band | |
| RR wt 2 | 1.1 | no | no | Faint band | |
| 401-1 | no | no | no | no | |
| 401-2 | 0.53 | no | no | no | |
| 401-3 | no | no | no | no | |
| Stevia | 0.58 | 1.8 | no | no | |

What is claimed is:

1. A method for modifying a plant, comprising expressing de novo or overexpressing at least one of 1-deoxy-D-xylulose 5-phosphate reductoisomerase (DXR), ent-copalyl diphosphate synthase (CPPS), and kaurenoic acid 13-hydroxylase (KAH), in a plant.

2. The method of claim 1, comprising transforming a plant with one or more expression cassettes that express at least one of the DXR, CPPS, and KAH genes, in the plant.

3. The method of claim 1, wherein the CPPS gene comprises either the DNA sequence of SEQ ID NO: 1, or encodes the protein of SEQ ID NO:2; wherein the DXR gene comprises either the DNA sequence of SEQ ID NO: 5, or encodes the protein of SEQ ID NO:6; and wherein the KAH gene comprises either the DNA sequence of SEQ ID NO: 9, or encodes the protein of SEQ ID NO:10.

4. The method of claim 1, comprising expressing de novo or overexpressing DXR, CPPS, and KAH in said plant.

5. The method of claim 1, comprising expressing de novo or overexpressing the DXR gene of Stevia rebaudiana in a plant other than Stevia rebaudiana.

6. The method of claim 1, comprising expressing de novo or overexpressing the CPPS gene of Stevia rebaudiana in a plant other than Stevia rebaudiana.

7. The method of claim 1, comprising expressing de novo or overexpressing the KAH gene of Stevia rebaudiana in a plant other than Stevia rebaudiana.

8. The method of claim 1, comprising stably integrating into the genome of at least one plant cell one or more exogenous genetic cassettes selected from the group consisting of (i) a gene expression cassette for expressing DXR, (ii) a gene expression cassette for expressing CPPS, and (iii) a gene expression cassette for expressing KAH.

9. The method of claim 1, wherein said plant produces at least 100% more kaurenoic acid than a wild plant of the same variety.

10. The method of claim 1, wherein the kaurenoic acid concentration in said plant is at least 10% of the kaurenoic acid concentration in a wild plant of Stevia rebaudiana.

11. A modified plant comprising in its genome one or more exogenous genetic cassettes selected from the group consisting of (i) a gene expression cassette for expressing DXR, (ii) a gene expression cassette for expressing CPPS, and (iii) a gene expression cassette for expressing KAH.

12. The plant of claim 11, comprising in its genome (i) a gene expression cassette for expressing DXR, (ii) a gene expression cassette for expressing CPPS, and (iii) a gene expression cassette for expressing KAH.

13. The plant of claim 11, wherein the CPPS gene either comprises the DNA sequence of SEQ ID NO: 1, or encodes the protein of SEQ ID NO:2; wherein the DXR gene either comprises the DNA sequence of SEQ ID NO: 5, or encodes the protein of SEQ ID NO:6; and wherein the KAH gene either comprises the DNA sequence of SEQ ID NO: 9, or encodes the protein of SEQ ID NO:10.

14. The plant of claim 11, wherein said plant is potato or strawberry.

15. The plant of claim 11, wherein said plant produces at least 100% more kaurenoic acid than a wild plant of the same variety, and wherein the kaurenoic acid concentration in said plant is at least 10% of the kaurenoic acid concentration in a wild plant of Stevia rebaudiana.

16. A food product or nutritional composition produced from the plant of claim 15.

17. A transformation vector, comprising one or more genetic cassettes selected from the group consisting of (i) a gene expression cassette for expressing DXR, (ii) a gene expression cassette for expressing CPPS, and (iii) a gene expression cassette for expressing KAH.

18. A method for up-regulating the expression of geranylgeranyl diphosphate synthase in a plant, comprising overexpressing or expressing de novo the DXR gene in said plant.

19. A method for producing kaurenoic acid in a plant, comprising overexpressing or expressing de novo the CPPS gene in said plant.

20. The method of claim 1, further comprising overexpressing or expressing de novo at least one glycosyltransferases in said plant.
